(19) United States
(12) Patent Application Publication
(10) Pub. No.: US 2025/0000905 A1
(43) Pub. Date: Jan. 2, 2025

(54) COMPOSITION FOR TREATING SOLID MALIGNANT TUMOR, AND KIT FOR TREATING SOLID MALIGNANT TUMOR

(71) Applicants: Kenichiro HASUMI, Tokyo (JP); Jun HASUMI, Tokyo (JP); CELL CO., LTD., Tokyo (JP)

(72) Inventor: Kenichiro HASUMI, Tokyo (JP)

(21) Appl. No.: 18/705,696

(22) PCT Filed: Oct. 24, 2022
(86) PCT No.: PCT/JP2022/039484

§ 371 Date: Apr. 29, 2024

Related U.S. Application Data
(60) Provisional application No. 63/275,576, filed on Nov. 4, 2021.

(30) Foreign Application Priority Data
Jul. 27, 2022 (JP) 2022-119776

(51) Int. Cl. A61K 35/17; A61K 31/573; A61K 39/00; A61P 35/00; C07K 16/24; C12N 5/00; C12N 5/0783; G01N 33/68

(52) U.S. Cl. CPC A61K 35/17; A61K 31/573; A61K 39/4611; A61K 39/4615; A61P 35/00; C07K 16/248; C12N 5/0018; C12N 5/0638; G01N 33/6893; A61K 2039/507; G01N 2333/525; G01N 2333/5409; G01N 2333/5412; G01N 2333/5421; G01N 2333/545

(57) ABSTRACT

Provided is a new therapeutic composition. A composition for treating a solid malignant tumor, the composition being used so as to be administered, in combination with at least one type selected from the group consisting of immature dendritic cells and cytotoxic lymphocytes induced by dendritic cells, to a subject having a malignant tumor cell that produces at least one type of inflammatory cytokine selected from the group consisting of TNFα, IL-1β, IL-5, IL-6, IL-8, IL-17, and IL-23, and the composition containing at least one type of antibody that inhibits action of the inflammatory cytokine.

### TECHNICAL FIELD

The present invention relates to a composition for treating a solid malignant tumor and a kit for treating a solid malignant tumor.

### BACKGROUND ART

As a cancer therapeutic method, cancer vaccine therapy using dendritic cells (hereinafter referred to as “dendritic cell vaccine therapy”) is known. In common dendritic cell vaccine therapy, immature dendritic cells collected from a patient are antigen-stimulated in vitro, and dendritic cells having undergone antigen recognition are administered subcutaneously to the patient or to lymph nodes of the patient to induce cytotoxic T cells (CTLs). Dendritic cell vaccine therapy is ordinarily used in combination with radiotherapy (e.g., Non-patent Literatures 1 to 4).

### CITATION LIST

### Non-Patent Literature

### [Non-patent Literature 1]

Jessica A Cintolo et. al., Dendritic cell-based vaccines: barriers and opportunities, Future Oncol. 2012 October; 8 (10): p. 1273-1299

### [Non-patent Literature 2]

Jifeng Yu et. al., Research progress on dendritic cell vaccines in cancer immunotherapy, Exp Hematol Oncol. 2022 Jan. 24; 11 (1): 3

### [Non-patent Literature 3]

Ivan Y. Filin et. al., Recent Advances in Experimental Dendritic Cell Vaccines for Cancer, Front Oncol. 2021 Sep. 23; 11

### [Non-patent Literature 4]

Beatris Mastelic-Gavillet et. al., Personalized Dendritic Cell Vaccines-Recent Breakthroughs and Encouraging Clinical Results, Front Immunol. 2019 Apr. 11; 10:766

### SUMMARY OF INVENTION

### Technical Problem

However, known dendritic cell vaccine therapy sometimes makes it impossible to sufficiently achieve an effect of regressing, reducing, or eliminating cancers. This requires development of a new cancer therapeutic method.

The present invention has been made in view of the above problem, and an object thereof is to provide a new therapeutic composition for treating a solid malignant tumor.

### Solution to Problem

In order to attain the object, the inventors of the present invention carried out diligent studies. As a result, the inventors of the present invention arrived at the present invention by finding that it is effective in treatment of a solid malignant tumor to, during administration of at least one type selected from the group consisting of immature dendritic cells and cytotoxic lymphocytes induced by dendritic cells to a subject having a malignant tumor cell that produces a given inflammatory cytokine, administer, in combination with these cells, at least one type of antibody that inhibits action of the inflammatory cytokine produced by the malignant tumor cell.

In order to attain the object, a composition for treating a solid malignant tumor in accordance with an aspect of the present invention is used so as to be administered, in combination with at least one type selected from the group consisting of immature dendritic cells and cytotoxic lymphocytes induced by dendritic cells, to a subject having a malignant tumor cell that produces at least one type of inflammatory cytokine selected from the group consisting of a tumor necrosis factor α, interleukin-1β, interleukin-5, interleukin-6, interleukin-8, interleukin-17, and interleukin-23, and the composition containing at least one type of antibody that inhibits action of the inflammatory cytokine.

A kit for treating a solid malignant tumor in accordance with an aspect of the present invention is used so as to be administered, in combination with at least one type selected from the group consisting of immature dendritic cells and cytotoxic lymphocytes induced by dendritic cells, to a subject having a malignant tumor cell that produces at least one type of inflammatory cytokine selected from the group consisting of a tumor necrosis factor α, interleukin-1β, interleukin-5, interleukin-6, interleukin-8, interleukin-17, and interleukin-23, and the kit including at least one type of antibody that inhibits action of the inflammatory cytokine.

### Advantageous Effects of Invention

An aspect of the present invention makes it possible to provide a new therapeutic composition for treating a solid malignant tumor.

### BRIEF DESCRIPTION OF DRAWINGS

FIG. 1 is a view illustrating Administration Example 1 in which a composition for treating a solid malignant tumor in accordance with an aspect of the present invention is administered.

FIG. 2 is a view illustrating Administration Example 2 in which the composition for treating a solid malignant tumor in accordance with an aspect of the present invention is administered.

FIG. 3 is a view illustrating Administration Example 3 in which the composition for treating a solid malignant tumor in accordance with an aspect of the present invention is administered.

FIG. 4 provides CT images that show a treatment result in Example 1.

FIG. 5 provides CT images that show a treatment result in Example 2.

FIG. 6 provides CT images that show a treatment result in Example 3.

FIG. 7 provides a CT image (drawing on the left) and an MRI image (drawing on the right) that show a treatment result in Example 4.

FIG. 8 provides CT images that show a treatment result in Example 5.

FIG. 9 provides PET images that show the treatment result in Example 2.

### DESCRIPTION OF EMBODIMENTS

### [1. Composition for Treating Solid Malignant Tumor]

### (Feature)

A composition for treating a solid malignant tumor (hereinafter also referred to merely as “therapeutic composition”) in accordance with an aspect of the present invention is used so as to be administered, in combination with at least one type selected from the group consisting of immature dendritic cells and cytotoxic lymphocytes induced by dendritic cells, to a subject having a malignant tumor cell that produces at least one type of inflammatory cytokine selected from the group consisting of a tumor necrosis factor α, interleukin-1β, interleukin-5, interleukin-6, interleukin-8, interleukin-17, and interleukin-23, and the composition containing at least one type of antibody that inhibits action of the inflammatory cytokine. In the present specification, “immature dendritic cell”, “dendritic cell”, and “cytotoxic T cell” are sometimes abbreviated as “iDC”, “DC”, and “CTL”, respectively.

In a case where a therapeutic composition in accordance with an aspect of the present invention is administered, in accordance with the above-described usage, in combination with at least one type selected from the group consisting of iDCs and CTLs induced by DCs, it is possible to expect an effect of regressing, reducing, or eliminating tumor cells in a solid malignant tumor tissue.

In a case where the therapeutic composition in accordance with an aspect of the present invention is administered, in accordance with the above-described usage, in combination with at least one type selected from the group consisting of iDCs and CTLs induced by DCs, an effect of regressing, reducing, or eliminating tumor cells in a solid malignant tumor tissue can be expected without use of radiotherapy in combination with the therapeutic composition. The therapeutic composition in accordance with an aspect of the present invention also has an advantage of causing fewer side effects. Thus, using the therapeutic composition in accordance with an aspect of the present invention makes it possible to provide a new cancer therapeutic method that is applicable also to a subject to which radiotherapy/chemotherapy has not been applicable due to a reason(s) such as age, the number of tumors, and/or the tumor size. Therefore, the therapeutic composition in accordance with an aspect of the present invention makes it possible to provide treatment of a solid malignant tumor to a wider range of subjects, as compared with a conventional therapeutic method. The therapeutic composition in accordance with an aspect of the present invention also contains at least one type of inflammatory cytokine blocking antibody that is in accordance with a type of inflammatory cytokine produced by a malignant tumor cell that a subject to which the therapeutic composition is to be administered has. Thus, the therapeutic composition in accordance with an aspect of the present invention makes it possible to provide a cancer therapeutic method that is suitable for a subject (is individualized). Such effects can contribute to, for example, Goal 3 “Ensure healthy lives and promote well-being for all at all ages” of Sustainable Development Goals (SDGs) proposed by the United Nations.

Note that the therapeutic composition in accordance with an aspect of the present invention may be used, if necessary, in combination with another treatment method useful for treating a target solid malignant tumor. The another treatment method is exemplified by, but not limited to, radiotherapy using X-rays, gamma rays, etc.; particle beam therapy; surgical treatment such as surgery; chemotherapy; and molecularly targeted therapy.

In the present specification, “treatment” includes (i) further regressing or reducing tumor cells in a solid malignant tumor tissue of a subject to which the therapeutic composition in accordance with an aspect of the present invention has been administered, than in the subject to which the therapeutic composition has not been administered, (ii) eliminating tumor cells in a solid malignant tumor tissue of the subject to which the therapeutic composition in accordance with an aspect of the present invention has been administered, or (iii) preventing solid malignant tumor progression in the subject to which therapeutic composition in accordance with an aspect of the present invention has been administered.

The therapeutic composition in accordance with an aspect of the present invention contains at least one type of antibody that inhibits action of at least one type of inflammatory cytokine produced by a tumor cell and selected from the group consisting of a tumor necrosis factor α, interleukin-1β, interleukin-5, interleukin-6, interleukin-8, interleukin-17, and interleukin-23. Thus, by inhibiting action of the inflammatory cytokine produced by the tumor cell, the antibody can prevent or reduce inflammation in a tumor that produces the inflammatory cytokine. Inflammation in a tumor tissue is considered to contribute to induction of a mutation in a tumor cell and promotion of tumor progression. Thus, elimination or reduction, or prevention of inflammation in a tumor tissue by the therapeutic composition in accordance with an aspect of the present invention is considered to bring about the above-described therapeutic effect.

The therapeutic composition in accordance with an aspect of the present invention also makes it possible to provide regression, reduction or elimination of tumor cells in tumor tissue which can be visually detected by MRI and/or CT and/or Echo scan.

In the present specification, “solid malignant tumor” means any malignant tumor, excluding hematological cancer. That is, the solid malignant tumor refers to any solid cancer and a malignant tumor in the brain. Examples of the solid malignant tumor include lung cancer, rectal cancer, uterine cancer, gastric cancer, and pancreatic cancer. From the viewpoint of a highly safe treatment, the therapeutic composition in accordance with an aspect of the present invention is preferably used to treat an elderly person and to treat a cancer to which standard treatment is not adapted. The solid malignant tumor includes both an early-stage cancer and an advanced cancer. The solid malignant tumor also includes a tumor formed by metastasis. In the present specification, “malignant tumor” means the solid malignant tumor.

### (Antibody)

The therapeutic composition in accordance with an aspect of the present invention contains at least one type of antibody that inhibits action of at least one type of inflammatory cytokine selected from the group consisting of a tumor necrosis factor α (TNFα), interleukin-1β (IL-1β), interleukin-5 (IL-5), interleukin-6 (IL-6), interleukin-8 (IL-8), interleukin-17 (IL-17), and interleukin-23 (IL-23). In the present specification, “inflammatory cytokine” refers to a cytokine that causes an inflammatory symptom in vivo. In the present specification, an antibody that inhibits action of an inflammatory cytokine is sometimes referred to as “inflammatory cytokine blocking antibody”, and an antibody that inhibits action of a specific inflammatory cytokine is sometimes referred to as, for example, “IL-6 blocking antibody”, “IL-5 blocking antibody”, or the like.

A type of the inflammatory cytokine blocking antibody is not particularly limited provided that the inflammatory cytokine blocking antibody is an antibody which specifically inhibits action of an inflammatory cytokine produced by a malignant tumor cell that a subject has. Examples of the inflammatory cytokine blocking antibody include all antibodies that can be used in pharmaceutical applications, such as polyclonal antibodies, monoclonal antibodies (e.g., IgG, IgM, IgE, IgA, IgD, and the like), modified antibodies (e.g., chimeric antibodies, humanized antibodies, and fully human antibodies), and antibody fragments (e.g., Fab, Fab′, F(ab′)₂, scFv, and the like).

The inflammatory cytokine blocking antibody is also referred to as “inflammatory cytokine neutralizing antibody”. The inflammatory cytokine blocking antibody may be, for example, an antibody that, by specifically binding to a target inflammatory cytokine (ligand), inhibits action of the inflammatory cytokine to which the antibody has bound, or may be an antibody that, by specifically binding to a receptor for a target inflammatory cytokine, inhibits action of the target inflammatory cytokine.

The inflammatory cytokine blocking antibody can be a commercially available antibody. Examples of such an antibody include Tocilizumab, which is a humanized anti-IL-6 receptor antibody, and Mepolizumab, which is a humanized anti-IL-5 receptor antibody.

The therapeutic composition in accordance with an aspect of the present invention need only contain at least one type of antibody that inhibits action of an inflammatory cytokine that is produced by a solid malignant tumor cell and that is among an inflammatory cytokine group consisting of TNFα, IL-1β, IL-5, IL-6, IL-8, IL-17, and IL-23. In a case where the therapeutic composition in accordance with an aspect of the present invention is used so as to be administered to a subject having a solid malignant tumor cell which produces a plurality of types of inflammatory cytokines among the inflammatory cytokine group consisting of TNFα, IL-1β, IL-5, IL-6, IL-8, IL-17, and IL-23, the therapeutic composition may contain a plurality of types of antibodies that inhibit action of the respective inflammatory cytokines. A method for screening inflammatory cytokines produced by a solid malignant tumor cell will be described later in [3. Method for treating solid malignant tumor].

In a case where the therapeutic composition in accordance with an aspect of the present invention is used so as to be administered to a subject having a solid malignant tumor cell that produces, for example, one or more types of inflammatory cytokines including IL-6, among the inflammatory cytokine group consisting of TNFα, IL-1β, IL-5, IL-6, IL-8, IL-17, and IL-23, the therapeutic composition preferably contains at least an IL-6 blocking antibody as an antibody that inhibits action of an inflammatory cytokine. The therapeutic composition in accordance with an aspect of the present invention is often configured to contain at least an IL-6 blocking antibody and an IL-5 blocking antibody. This is because IL-6 and IL-5 are expressed intratumorally at a high rate and particularly greatly contribute to inflammation. Thus, a therapeutic composition containing at least the IL-6 blocking antibody and the IL-5 blocking antibody makes it possible to effectively eliminate or reduce, or prevent inflammation caused by an immunoresponse in a malignant tumor tissue. As a result, tumor cells in a solid malignant tumor tissue can be regressed, reduced, or eliminated. However, in a case where the therapeutic composition in accordance with an aspect of the present invention is used so as to be administered to a subject having a solid malignant tumor cell that does not produce IL-6 or IL-5, the therapeutic composition need not contain the IL-6 blocking antibody or the IL-5 blocking antibody as the antibody that inhibits action of an inflammatory cytokine.

In a case where the therapeutic composition in accordance with an aspect of the present invention is used so as to be administered to a subject having a solid malignant tumor cell that produces two or more types of inflammatory cytokines including IL-6 and IL-5, among the inflammatory cytokine group consisting of TNFα, IL-1β, IL-5, IL-6, IL-8, IL-17, and IL-23, the therapeutic composition preferably contains at least the IL-5 blocking antibody and the IL-6 blocking antibody as the antibody that inhibits action of an inflammatory cytokine. As described earlier, IL-6 and IL-5 are expressed intratumorally at a high rate and greatly contribute to inflammation. A therapeutic composition containing the IL-6 blocking antibody and the IL-5 blocking antibody makes it possible to suppress inflammation caused by both IL-5 and IL-6. It is therefore possible to effectively eliminate or reduce, or prevent inflammation in a malignant tumor tissue. As a result, tumor cells in a solid malignant tumor tissue can be regressed, reduced, or eliminated.

Assume that the therapeutic composition in accordance with an aspect of the present invention is used so as to be administered to a subject for which it is difficult to determine a type of cytokine produced by a solid malignant tumor cell. In this case, the therapeutic composition in accordance with an aspect of the present invention preferably contains, as the antibody that inhibits action of an inflammatory cytokine, at least the IL-5 blocking antibody and the IL-6 blocking antibody in a case where the type of the cytokine produced by the solid malignant tumor cell is unknown but at least one type of inflammatory cytokine among the inflammatory cytokine group consisting of TNFα, IL-1β, IL-5, IL-6, IL-8, IL-17, and IL-23 can be expected to be produced.

The inventors of the present invention found that an expression rate of an inflammatory cytokine in a solid malignant tumor cell is approximately 90% for TNFα and IL-1B and that a positive rate tends to be lower in descending order of IL-6, IL-5, IL-8, IL-23, and IL-17. However, in a case where the therapeutic composition in accordance with an aspect of the present invention is used so as to be administered to a subject having a solid malignant tumor cell in which such a tendency in inflammatory cytokine expression is observed, the therapeutic composition need only contain at least one type of antibody that inhibits action of an inflammatory cytokine produced by a solid malignant tumor cell, among an inflammatory cytokine group consisting of IL-6, IL-5, IL-8, IL-23, and IL-17. It is also possible to, without administering any antibody that inhibits action of TNFα, administer, in any combination, antibodies that inhibit action of inflammatory cytokines other than TNFα.

The therapeutic composition in accordance with an aspect of the present invention contains the inflammatory cytokine blocking antibody as an active ingredient. The therapeutic composition in accordance with an aspect of the present invention may contain an active ingredient other than the inflammatory cytokine blocking antibody. The active ingredient(s) is/are contained in the therapeutic composition in accordance with an aspect of the present invention in an amount that is not particularly limited and is, for example, 0.001% by weight to 100% by weight, 0.01% by weight to 100% by weight, 0.1% by weight to 100% by weight, 0.1% by weight to 95% by weight, 0.1% by weight to 90% by weight, 0.1% by weight to 80% by weight, 0.1% by weight to 70% by weight, 0.1% by weight to 60% by weight, 0.1% by weight to 50% by weight, 0.1% by weight to 40% by weight, 0.1% by weight to 30% by weight, 0.1% by weight to 20% by weight, and 0.1% by weight to 10% by weight, with respect to a total weight of the therapeutic composition in accordance with an aspect of the present invention.

### (Other Ingredient(s))

The therapeutic composition in accordance with an aspect of the present invention may contain, if necessary, a component other than the foregoing inflammatory cytokine blocking antibody. An other ingredient(s) need only be a pharmaceutically acceptable ingredient(s) and can be, for example, any of a buffer, a pH regulator, a tonicity agent, an antiseptic, an antioxidant, a high molecular weight polymer, a vehicle, and a solvent. Such an ingredient need only be a substance that is commonly contained in a therapeutic composition, the type of which is not particularly limited.

The therapeutic composition in accordance with an aspect of the present invention may contain an effective ingredient having a desired effect as the foregoing other ingredient. Examples of the desired effect include an effect of reducing a side effect and an effect of helping suppression of inflammation.

The other ingredient(s) is/are contained in the therapeutic composition in accordance with an aspect of the present invention in an amount that is not particularly limited and is, for example, 0% by weight to 99.999% by weight, 0% by weight to 99.99% by weight, 0% by weight to 99.9% by weight, 5% by weight to 99.9% by weight, 10% by weight to 99.9% by weight, 20% by weight to 99.9% by weight, 30% by weight to 99.9% by weight, 40% by weight to 99.9% by weight, 50% by weight to 99.9% by weight, 60% by weight to 99.9% by weight, 70% by weight to 99.9% by weight, 80% by weight to 99.9% by weight, and 90% by weight to 99.9% by weight, with respect to the total weight of the therapeutic composition in accordance with an aspect of the present invention.

### (Formulation and Dosage Form)

The therapeutic composition in accordance with an aspect of the present invention can be formulated by a known method by using, as materials, the inflammatory cytokine blocking antibody, serving as the active ingredient, and the other ingredient(s).

A dosage form of the therapeutic composition in accordance with an aspect of the present invention is not particularly limited but is preferably a liquid pharmaceutical formulation because the liquid pharmaceutical formulation is easily administered into a tumor or a blood vessel in a subject. Examples of the dosage form include a formulation for injection.

### (Usage)

The therapeutic composition in accordance with an aspect of the present invention is used so as to be administered, in combination with at least one type selected from the group consisting of iDCs and CTLs induced by DCs, to a subject having a malignant tumor cell that produces at least one type of inflammatory cytokine selected from the group consisting of TNFα, IL-1B, IL-5, IL-6, IL-8, IL-17, and IL-23. By being administered to the subject in combination with the at least one type selected from the group consisting of iDCs and CTLs induced by DCs, the therapeutic composition in accordance with an aspect of the present invention makes it possible to eliminate or reduce, or prevent inflammation caused by an immunoresponse in a malignant tumor. As a result, tumor cells in a solid malignant tumor tissue can be regressed, reduced, or eliminated.

In the present specification, a timing at which the at least one type selected from the group consisting of iDCs and CTLs induced by DCs is administered in combination with the therapeutic composition is not particularly limited. For example, (i) the inflammatory cytokine blocking antibody may be administered simultaneously with the at least one type selected from the group consisting of iDCs and CTLs induced by DCs, (ii) the inflammatory cytokine blocking antibody may be administered, at predetermined intervals, before or after administration of the at least one type selected from the group consisting of iDCs and CTLs induced by DCs, or (i) and (ii) may be combined. In order to effectively inhibit inflammation caused by CTLs induced by DCs, it is preferable to administer the therapeutic composition in accordance with an aspect of the present invention simultaneously with the at least one type selected from the group consisting of iDCs and CTLs induced by DCs.

In a case where the at least one type selected from the group consisting of iDCs and CTLs induced by DCs and the therapeutic composition are administered at intervals, the therapeutic composition is preferably administered within 90 days after administration of the at least one type selected from the group consisting of iDCs and CTLs induced by DCs, in order to suppress a change to an inflammatory tumor.

In the present specification, “immature dendritic cells” or “iDCs” refer to dendritic cells that are not antigen-stimulated and that have phagocytic activity of antigens. The immature dendritic cells include a dendritic cell population in which myeloid markers CD11c and CD14 are positive, costimulatory markers CD14, CD86, and HLA-DR are positive, and a dendritic cell maturation marker CD83 is negative. The immature dendritic cells are not particularly limited in origin. In order to prevent rejection, it is possible to suitably use autologous immature dendritic cells obtained from a subject. Autologous immature dendritic cells can be prepared by, for example, a method for culturing monocyte cell fractions of peripheral blood mononuclear cells (PBMCs) collected from a subject, a method for obtaining iDCs from hematopoietic stem cells collected from a subject, or a method for obtaining iDCs by apheresis from a subject.

By being intratumorally administered, the immature dendritic cells are sensitized in a tumor, e.g., at a tumor site, and present, to cell surfaces, comprehensive tumor antigens including both known and unknown tumor antigens. Dendritic cells that have presented tumor antigens activate T cells to induce tumor antigen-specific CTLs.

iDCs whose maturation is being induced with an adjuvant added before administration may be administered in combination with the therapeutic composition in accordance with an aspect of the present invention. The adjuvant can include, without limitation, lipid-based, protein-based and polysaccharides-based adjuvants, such as lymphocyte culture medium, Marignase, Agaricus, OK432, BCG, Lentinan (shiitake), Reishi, Sarunokoshikake, TNF Meshimakobu, Freund's complete or incomplete adjuvant, LPS, fatty acids, TW80, phospholipids, cytokines or a virus. In certain embodiments, the adjuvant can be a leukocyte cultured medium (LCM) adjuvant. The LCM adjuvant can include at least three cytokines selected from the group consisting of eotaxin, FGF, G-CSF, GM-CSF, IFNγ, IP10, IL-1β, IL-1ra, IL-2, IL-4, IL-5, IL-6, IL-7, IL-8, IL-9, IL-10, IL-12, IL-13, IL-15, IL-17, MCP1, MIP1α, MIP1β, PDGFbb, RANTES, TNFα and VEGF.

In the present specification, “cytotoxic lymphocytes” or “CTLs” are cytotoxic lymphocytes induced by dendritic cells. The CTLs include a CTL induced by an immature dendritic cell that has been administered to a subject and intratumorally sensitized and matured. It is possible to suitably use, as the CTLs, autologous CTLs obtained from a subject. The autologous CTLs can be prepared by culturing T-cell enriched fractions of peripheral blood mononuclear cells (PBMCs) collected from a subject. Alternatively, the CTLs may be CTLs induced and prepared by artificially sensitizing T cells in vitro to limited known-antigens. In this case, the CTLs are effective in malignant tumors that respond to these limited antigens.

Cytotoxic T lymphocytes (CTLs) are an important component of cellular immunity. They play a critical role in the control of many infections and cancers. These T cells are responsible for “hunting down” other cells of the body that are infected by viruses or are cancer-containing, and destroying them. For example, when a virus or cancer is using a cell to reproduce, the cell displays some of the viral proteins or cancer components on its surface. The cytotoxic T cells recognize these proteins or components and hone-in to destroy the infected or cancer-containing cells before they can release the new infection or cancer into the bloodstream. Many vaccines are effective, at least in part, by stimulating this type of T cell activation or response. CTLs can also create chemicals known as cytokines which assist in coordinating how the immune system fights against disease.

The therapeutic composition in accordance with an aspect of the present invention is preferably used so as to be administered in combination with activated T cells (ATs). The ATs include a cell population in which lymphocyte markers CD3 and CD4 are positive, and T cell activation markers CD25 and CD154 (CD40L) are positive. It is possible to suitably use, as the ATs, autologous AT cells obtained from a subject. The autologous ATs can be prepared by culturing T-cell enriched fractions of peripheral blood mononuclear cells (PBMCs) collected from a subject. Administration of the ATs in combination with the therapeutic composition in accordance with an aspect of the present invention results in efficient production of the CTLs and consequently makes it possible to enhance a reducing effect on a solid malignant tumor. Further, an AT by which CD154 is artificially expressed acts as a preCTL. The AT by which CD154 is artificially expressed changes into a matured CTL after receiving antigen information from a DC, and forms an immunological memory. Thus, administration of the ATs in combination with the therapeutic composition in accordance with an aspect of the present invention enables efficient production of the CTLs.

The therapeutic composition in accordance with an aspect of the present invention is preferably used so as to be administered in combination with an anti-inflammatory agent other than the inflammatory cytokine inhibitory antibody. The anti-inflammatory agent is not particularly limited, and any anti-inflammatory agent that can be used in a pharmaceutical application can be used. Examples of such an anti-inflammatory agent include adrenocorticosteroids and non-steroidal antiphlogistics. Examples of the adrenocorticosteroids include dexamethasone, prednisolone, clobetasol propionate, betamethasone dipropionate, and hydrocortisone butyrate. The therapeutic composition in accordance with an aspect of the present invention is preferably used so as to be administered to a subject in combination with dexamethasone. This is because it is possible to expect not only an anti-inflammatory effect but also an appetite-stimulating effect and an antiemetic effect. Further, assume that the therapeutic composition in accordance with an aspect of the present invention is used so as to be administered to a subject having a malignant tumor cell that produces at least one type of inflammatory cytokine selected from the group consisting of IL-17 and IL-23 among the inflammatory cytokine group consisting of TNFα, IL-1β, IL-5, IL-6, IL-8, IL-17, and IL-23. In this case, in cost terms, it is possible to administer an adrenocorticosteroid instead of administering, to the subject, at least one type selected from the group consisting of an IL-17 blocking antibody and an IL-23 blocking antibody.

Dexamethasone may be in the form of “pharmaceutically acceptable salt”. That is, in the present specification, the concept of dexamethasone also includes a pharmaceutically acceptable salt. In the present specification, “pharmaceutically acceptable salt” means a salt the administration of which as a pharmaceutical to a subject is physiologically acceptable, and a specific example thereof is not limited. Examples of such a salt include alkali metal salts (such as a potassium salt), alkaline earth metal salts (such as a calcium salt and a magnesium salt), ammonium salts, organic basic salts (such as a trimethylamine salt, a triethylamine salt, a pyridine salt, a picoline salt, a dicyclohexylamine salt, and N,N′-dibenzylethylenediamine salt), organic acid salts (such as acetate, maleate, tartrate, methanesulfonate, benzenesulfonate, formate, toluene sulfonate, and trifluoroacetate), and inorganic acid salts (such as hydrochloride, hydrobromide, sulfate, and phosphate).

The therapeutic composition in accordance with an aspect of the present invention may be administered in combination with a chemical other than an antibody that inhibits action of TNFα.

The therapeutic composition in accordance with an aspect of the present invention can be administered to a subject by any administration route. Examples of the administration route include intraarterial administration, intravenous administration, intramuscular administration, intraperitoneal administration, intratumoral administration, intrapleural administration, and subcutaneous administration. The therapeutic composition in accordance with an aspect of the present invention is preferably administered into a malignant tumor in a subject because it is easier to obtain an effect.

As used herein, the term “intravenously”, “through vessel” and related terms employing “through vessel” refer to therapy that includes the introduction of components which can be introduced into a subject and combinations thereof, into a channel, such as, a vein or artery, in a patient's body for carrying fluid.

As used herein, the term “intratumoral therapy” and related terms employing “intratumoral” or “intratumorally” refer to therapy that includes the introduction of components which can be introduced into a subject and combinations thereof, directly into the tumor tissue of a patient.

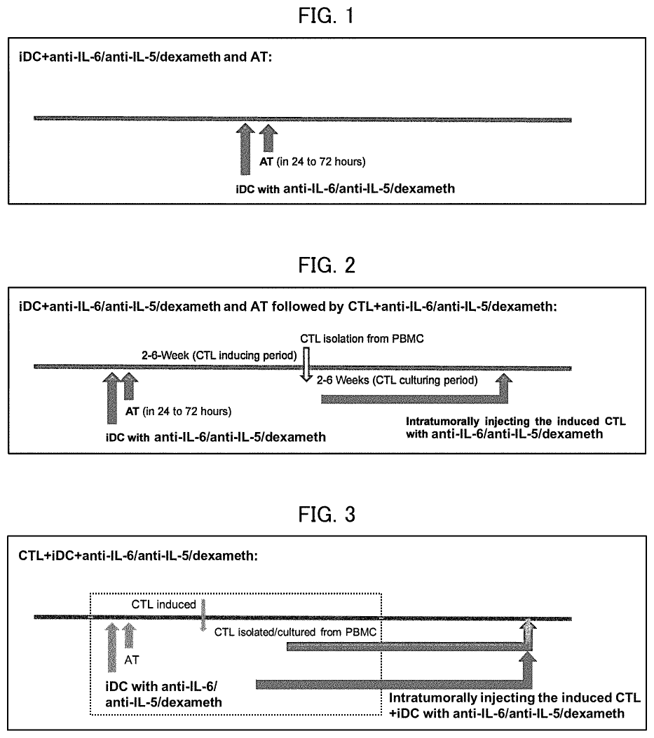

The following description will discuss an example of usage of a therapeutic composition with reference to FIGS. 1 to 3. In each of Administration Examples 1 to 3 described below, the therapeutic composition containing, as the inflammatory cytokine blocking antibody, the IL-6 blocking antibody and the IL-5 blocking antibody is administered to a subject. Note, however, that the inflammatory cytokine blocking antibody that can be used in the therapeutic composition in accordance with an aspect of the present invention is not limited to inflammatory cytokine inhibiting antibodies used in Administration Examples 1 to 3. For example, the IL-6 blocking antibody alone may be used.

FIG. 1 is a view illustrating Administration Example 1 in which the therapeutic composition is administered. First, the therapeutic composition is administered to the subject simultaneously with iDCs and dexamethasone. Subsequently, ATs are administered in 24 to 72 hours. In a therapeutic protocol shown in FIG. 1, administration of the iDCs makes it possible to induce comprehensive tumor antigen-specific CTLs containing both known and unknown tumor antigens. In the therapeutic protocol shown in FIG. 1, the therapeutic composition is administered simultaneously with administration of the iDCs and the dexamethasone. With such usage, inflammation in a solid malignant tumor tissue caused by an immunoresponse by the CTLs induced by administration of the iDCs can be eliminated or reduced, or prevented by the therapeutic composition. As a result, tumor cells in the solid malignant tumor tissue can be effectively regressed, reduced, or eliminated.

FIG. 2 is a view illustrating Administration Example 2 in which the therapeutic composition is administered. First, the therapeutic composition is administered to the subject simultaneously with iDCs and dexamethasone. Subsequently, ATs are administered in 24 to 72 hours. After a CTL producing period of 2 to 6 weeks, CTLs are isolated from peripheral blood mononuclear cells, and the CTLs are cultured for 2 to 6 weeks. Subsequently, the therapeutic composition is intratumorally administered simultaneously with the induced CTLs and the dexamethasone. In a therapeutic protocol shown in FIG. 2, the CTLs induced in a body of the subject by administration of the iDCs are collected from the subject, and the collected CTLs are cultured in vitro and reintroduced into the subject. This makes it possible to introduce, into the subject, a sufficient amount (e.g., therapeutically effective amount) of CTLs to effectively regress, reduce, or eliminate tumor cells in a solid malignant tumor tissue.

In the therapeutic protocol shown in FIG. 2, the therapeutic composition is administered simultaneously with administration of the iDCs and the dexamethasone, and simultaneously with administration of the induced CTLs and the dexamethasone. With such usage, inflammation in a solid malignant tumor tissue caused by an immunoresponse by the induced CTLs can be eliminated or reduced, or prevented by the therapeutic composition. As a result, tumor cells in the solid malignant tumor tissue can be effectively regressed, reduced, or eliminated.

FIG. 3 is a view illustrating Administration Example 3 in which the therapeutic composition is administered. First, the therapeutic composition is administered to the subject simultaneously with iDCs and dexamethasone. Subsequently, ATs are administered. After CTLs are induced, the CTLs are isolated from peripheral blood mononuclear cells and cultured. Subsequently, the induced CTLs, the iDCs, the dexamethasone, and the therapeutic composition are intratumorally administered. In a therapeutic protocol shown in FIG. 3, the iDCs can take up tumor cells that are impaired by the induced CTLs. In addition, the CTLs can be upgraded by administering the iDCs, the induced CTLs, and the dexamethasone. This makes it possible to treat (i) malignant tumor cells that were not able to be regressed, reduced, or eliminated by first administration or (ii) tumor cells in a tumor tissue newly formed by metastasis.

In the therapeutic protocol shown in FIG. 3, the therapeutic composition is administered simultaneously with administration of the iDCs and the dexamethasone, and simultaneously with administration of the induced CTLs and the dexamethasone. With such usage, inflammation in a solid malignant tumor tissue caused by an immunoresponse by the induced CTLs can be eliminated or reduced, or prevented by the therapeutic composition. As a result, tumor cells in the solid malignant tumor tissue can be effectively regressed, reduced, or eliminated.

In the treatment protocols shown in FIGS. 2 and 3, after at least 6 months have passed since the first administration of the therapeutic composition, the iDCs, and the dexamethasone, the iDCs, the induced CTLs, the dexamethasone, and the therapeutic composition may be administered into a tumor having been subjected to the first administration and another tumor (e.g., a new focus). The CTLs can be upgraded by administering the iDCs, the induced CTLs, and the dexamethasone to a new focus in the case of progression of a mutation in a tumor cell. The therapeutic composition is administered simultaneously with administration of the iDCs, the induced CTLs, and the dexamethasone. With such usage, inflammation in a solid malignant tumor tissue caused by an immunoresponse by the induced CTLs can be eliminated or reduced, or prevented by the therapeutic composition. As a result, tumor cells in the solid malignant tumor tissue can be effectively regressed, reduced, or eliminated.

As an example of such a therapeutic protocol, it is possible to, for example, (i) administer the iDCs, the therapeutic composition, and the dexamethasone, (ii) subsequently (for example, after 2 to 4 months, and preferably after 3 months), administer the CTLs, the therapeutic compositions, and the dexamethasone, and (iii) further subsequently (for example, after 2 to 4 months, and preferably after 3 months), administer the iDCs, the CTLs, the therapeutic composition, and the dexamethasone to a new focus. In this case, a degree of achievement of treatment in each administration stage can be approximately 50% for first administration, approximately 80% for second administration, and approximately 100% for third administration (complete remission).

In the therapeutic protocol shown in FIG. 3, the therapeutic composition, the iDCs, and the dexamethasone may be repeatedly administered after a certain period of time has passed since the first administration of the therapeutic composition, the iDCs, and the dexamethasone. Repeated administration of the therapeutic composition, the iDCs, and the dexamethasone makes it possible to repeatedly treat (i) malignant tumor cells that were not able to be regressed, reduced, or eliminated by the first administration or (ii) tumor cells in a tumor tissue newly formed by metastasis.

### (Dose)

In a case where the therapeutic composition in accordance with an aspect of the present invention is administered to a subject, an amount of administration of the therapeutic composition to the subject is not limited provided that a desired effect is obtained. For example, the therapeutic composition in accordance with an aspect of the present invention may be administered such that the inflammatory cytokine blocking antibody is administered in an amount of 0.1 mg/kg body weight to 1000.0 mg/kg body weight, 0.1 mg/kg body weight to 500.0 mg/kg body weight, 1.0 mg/kg body weight to 500.0 mg/kg body weight, 1.0 mg/kg body weight to 300.0 mg/kg body weight, 1.0 mg/kg body weight to 100.0 mg/kg body weight, 1.0 mg/kg body weight to 50.0 mg/kg body weight, 1.0 mg/kg to 10.0 mg/kg, 1.0 mg/kg body weight to 10.0 mg/kg body weight, or 1.0 mg/kg body weight to 5.0 mg/kg body weight.

### (Subject)

The subject has a malignant tumor cell that produces at least one type of inflammatory cytokine selected from the group consisting of TNFα, IL-1β, IL-5, IL-6, IL-8, IL-17, and IL-23.

As used herein, “subject” includes mammals including a human. In the present specification, “subject” is sometimes referred to as “patient”. The subject is preferably a mammal, and more preferably a human. Examples of a non-human mammal include companion animals such as a dog and a cat.

### [2. Kit for Treating Solid Malignant Tumor]

### (Feature)

A kit for treating a solid malignant tumor in accordance with an aspect of the present invention (hereinafter also referred to merely as “therapeutic kit”) is used so as to be administered, in combination with at least one type selected from the group consisting of immature dendritic cells and cytotoxic lymphocytes induced by dendritic cells, to a subject having a malignant tumor cell that produces at least one type of inflammatory cytokine selected from the group consisting of a tumor necrosis factor α, interleukin-1β, interleukin-5, interleukin-6, interleukin-8, interleukin-17, and interleukin-23, and the kit including at least one type of antibody that inhibits action of the inflammatory cytokine.

Since an effect, etc. of the kit are as have been described for the composition for treating a solid malignant tumor in accordance with an aspect of the present invention, a description thereof is omitted here.

A therapeutic kit in accordance with an aspect of the present invention includes at least one type of inflammatory cytokine blocking antibody. A user may mix blocking antibodies and administer the blocking antibodies to a subject, and may administer the blocking antibodies to the subject at different timings. Descriptions of the inflammatory cytokine blocking antibody and of usage and a dose of the inflammatory cytokine blocking antibody are identical to the descriptions in [1. Composition for treating solid malignant tumor].

The therapeutic kit in accordance with an aspect of the present invention may include, if necessary, a composition other than the inflammatory cytokine blocking antibody. Examples of the composition include a reagent other than the inflammatory cytokine blocking antibody, an instrument, and an instruction manual for the therapeutic kit.

A description of a component other than the inflammatory cytokine blocking antibody is identical to the description in [1. Composition for treating solid malignant tumor].

Examples of the instrument include an instrument for preparing a reagent and an instrument for administration to the subject.

The instruction manual for the therapeutic kit may include, for example, (i) the usage and the dose that have been described in [1. Composition for treating solid malignant tumor] and/or (ii) an example of a specific procedure for the therapeutic method described in [3. Method for treating solid malignant tumor] described later.

### [3. Method for Treating Solid Malignant Tumor]

### (Feature)

A method for treating a solid malignant tumor in accordance with an aspect of the present invention (hereinafter referred to merely as “therapeutic method”) includes the following (i) step (1) and (ii) at least one selected from the group consisting of (a) step (2) and (b) steps (3) to (5) of:
(1) screening, from the group consisting of a tumor necrosis factor α, interleukin-1β, interleukin-5, interleukin-6, interleukin-8, interleukin-17, and interleukin-23, an inflammatory cytokine produced by the malignant tumor cell that a subject has;
(2) administering, to the subject, immature dendritic cells and at least one type of antibody that inhibits action of the inflammatory cytokine screened through the step (1);
(3) collecting peripheral blood mononuclear cells from the subject;
(4) culturing the collected peripheral blood mononuclear cells and forming cytotoxic lymphocytes induced by dendritic cells; and
(5) administering, to the subject, the formed cytotoxic lymphocytes and the at least one type of antibody that inhibits action of the inflammatory cytokine screened through the step (1).

According to the therapeutic method in accordance with an aspect of the present invention, an effect of regressing, or eliminating tumor cells in a solid malignant tumor tissue can be expected without use of radiotherapy in combination with the therapeutic method. The therapeutic method in accordance with an aspect of the present invention also has an advantage of causing fewer side effects. Examples of a side effect caused by radiotherapy include mutation that is potentially induced and unknown mutations that occur when malignant tumor cells are lysed by irradiation. Thus, the therapeutic method in accordance with an aspect of the present invention makes it possible to provide a new cancer therapeutic method that is applicable also to a subject to which radiotherapy/chemotherapy has not been applicable due to a reason(s) such as age, the number of tumors, and/or the tumor size. Therefore, the therapeutic method in accordance with an aspect of the present invention makes it possible to provide treatment of a solid malignant tumor to a wider range of subjects, as compared with a conventional therapeutic method.

Note that the therapeutic method in accordance with an aspect of the present invention may be used, if necessary, in combination with another treatment method useful for treating a target solid malignant tumor. The another treatment method is exemplified by, but not limited to, radiotherapy using X-rays, gamma rays, etc.; particle beam therapy; surgical treatment such as surgery; chemotherapy; and molecularly targeted therapy.

In the therapeutic method in accordance with an aspect of the present invention, at least one type of antibody that inhibits action of at least one type of inflammatory cytokine produced by a tumor cell and selected from the group consisting of TNFα, IL-1β, IL-5, IL-6, IL-8, IL-17, and IL-23 is contained. Thus, by inhibiting, neutralizing, or blocking action of the inflammatory cytokine produced by the tumor cell, the antibody can prevent or reduce inflammation in a tumor that produces the inflammatory cytokine. Inflammation in a tumor tissue is considered to contribute to induction of a mutation in a tumor cell and promotion of tumor progression. Thus, elimination or reduction, or prevention of inflammation in a tumor tissue by the therapeutic method in accordance with an aspect of the present invention is considered to bring about the above-described therapeutic effect.

The therapeutic method in accordance with an aspect of the present invention also makes it possible to provide regression, reduction or elimination of tumor cells in tumor tissue which can be visually detected by MRI and/or CT and/or Echo scan.

A description of a subject to which the therapeutic method in accordance with an aspect of the present invention is to be applied is identical to the description in [1. Composition for treating solid malignant tumor].

In the therapeutic method in accordance with an aspect of the present invention, administration can be carried out with respect to a subject by any administration route. A description of an example of the administration route is identical to the description of the administration route in [1. Composition for treating solid malignant tumor]. In the therapeutic method in accordance with an aspect of the present invention, the administration route is preferably administration into a malignant tumor in a subject because it is easier to obtain an effect.

The therapeutic method in accordance with an aspect of the present invention may include the steps (1) and (2), the steps (1) and (3) to (5), or the steps (1) to (5).

In a case where the therapeutic method in accordance with an aspect of the present invention includes the steps (1) and (2), the iDCs; and the at least one type of antibody that inhibits action of the inflammatory cytokine produced by the malignant tumor cell in the subject, among the inflammatory cytokine group consisting of TNFα, IL-1β, IL-5, IL-6, IL-8, IL-17, and IL-23 can be administered to the subject.

In a case where the therapeutic method in accordance with an aspect of the present invention includes the steps (1) and (3) to (5), the CTLs induced by DCs; and the at least one type of antibody that inhibits action of the inflammatory cytokine produced by the malignant tumor cell in the subject, among the inflammatory cytokine group consisting of TNFα, IL-1β, IL-5, IL-6, IL-8, IL-17, and IL-23 can be administered to the subject.

In a case where the therapeutic method in accordance with an aspect of the present invention includes the steps (1) to (5), the iDCs; the at least one type of antibody that inhibits action of the inflammatory cytokine produced by the malignant tumor cell in the subject, among the inflammatory cytokine group consisting of TNFα, IL-1β, IL-5, IL-6, IL-8, IL-17, and IL-23; and the CTLs induced by DCs can be administered to the subject.

In a case where the therapeutic method in accordance with an aspect of the present invention includes the steps (1) to (5), the steps (3) to (5) may be carried out after the step (2), or the steps (2) and (5) may be carried out simultaneously. In a case where the steps (3) to (5) are carried out after the step (2), resulting CTLs can include the CTLs induced by DCs obtained by maturation of the administered iDCs. That is, by administering iDCs derived from patients into tumor tissues where all known and unknown-antigens (neoantigen) coexist, an aspect of the present invention makes it possible to produce rather, e.g., significantly, potent comprehensive CTLs that can respond to a larger number of tumors in the natural human immune functions.

As used herein, “comprehensive” CTLs refer to both known and unknown antigen-driven CTLs, e.g., each CTL has independence/specificity to each antigen.

### (Step (1))

The step (1) is a step of screening, from the group consisting of TNFα, IL-1β, IL-5, IL-6, IL-8, IL-17, and IL-23, an inflammatory cytokine produced by the malignant tumor cell that a subject has. A screening method is not particularly limited. Screening can be carried out by a known method that makes it possible to screen an inflammatory cytokine. Examples of a qualitative screening method include a method in which an antibody that specifically recognizes each cytokine is used to subject a malignant tumor cell to immunostaining so as to determine presence/absence of inflammatory cytokine protein expression in a tumor cell. Instead of determining presence/absence of inflammatory cytokine expression, it is possible to determine presence/absence of inflammatory cytokine receptor protein expression in a tumor cell. Further, examples of a quantitative screening method include a method in which an amount of blood inflammatory cytokine secreted from a tumor cell is measured.

In order to determine presence/absence of inflammatory cytokine expression in a malignant tumor, it is only necessary to carry out screening for any malignant tumor among malignant tumors to be treated. It is preferable to collect cells from at least two parts of any malignant tumor and carry out screening.

Further, screening need only be carried out for at least one type among the above-listed inflammatory cytokines, and a plurality of types may be selected and screened. As described earlier, an expression rate of an inflammatory cytokine in a solid malignant tumor cell is approximately 90% for TNFα and IL-1β, and a positive rate tends to be lower in descending order of IL-6, IL-5, IL-8, IL-23, and IL-17. Thus, in order to achieve more efficient screening, it is possible to determine presence/absence of expression preferentially from a cytokine having a high positive rate. IL-6 and IL-5 are expressed intratumorally at a high rate and particularly greatly contribute to inflammation. Thus, at least one type selected from the group consisting of IL-6 and IL-5 is preferably screened first.

The step (1) may be carried out at any timing provided that the timing is before an antibody which inhibits action of the screened inflammatory cytokine is administered to the subject. Further, in a case where the antibody which inhibits action of the screened inflammatory cytokine is to be administered to the subject a plurality of times, screening need only be carried out at least one time. Alternatively, screening may be carried out every time before administration of the antibody. For example, after an inflammatory cytokine produced by any malignant tumor is screened and the step (2) or (5) is carried out, an inflammatory cytokine produced by (i) a malignant tumor that has not been regressed, decreased, or eliminated, or (ii) a metastasized malignant tumor may be screened.

### (Step (2))

The step (2) is a step of administering, to the subject, iDCs and at least one type of antibody that inhibits action of the inflammatory cytokine screened through the step (1). According to the step (2), by being administered to the subject, the iDCs are sensitized in a tumor, e.g., at a tumor site, and present, to cell surfaces, comprehensive tumor antigens including both known and unknown tumor antigens. Dendritic cells that have presented tumor antigens activate T cells to induce tumor antigen-specific CTLs. Thus, the introduction of iDCs can positively promote an immunoresponse to induce comprehensive CTLs. A description of the iDCs is identical to the description of the iDCs in [1. Composition for treating solid malignant tumor].

In the step (2), the iDCs and an inflammatory cytokine blocking antibody may be administered simultaneously. Alternatively, administration of at least one type selected from the group consisting of the iDCs and the inflammatory cytokine blocking antibody may follow or be followed by administration of one of the iDCs and the inflammatory cytokine blocking antibody, which one has not been administered, at predetermined intervals.

Further, the iDCs and the inflammatory cytokine blocking antibody can be combined to form a composition, and the composition can be introduced intratumorally into the patient.

An amount of the iDCs introduced can be a therapeutically effective amount for a solid malignant tumor. As used herein, the term “therapeutically effective amount” refers to an amount of a component having an effect of treating a solid malignant tumor, required to bring about a desired effect in a human or other mammal. In the present specification, “a component having an effect of treating a solid malignant tumor” refers to iDCs, ATs, comprehensive CTLs, an inflammatory cytokine blocking antibody, dexamethasone, anti-inflammatory agent, adjuvant, or combinations thereof. In all instances, at its most basic level, the desired effect is a regression, reduction, or elimination of tumor cells in tumor tissue of the patient when compared to the tumor cells in the tumor tissue of the patient prior to employing the therapy and methods of an aspect of the present invention.

The iDCs can be administered, for example, once to the subject, such that, for example, 5×10⁶ iDCs to 1×10⁷ iDCs per tumor are administered. Further, the amount of the iDCs administered can be adjusted as appropriate in accordance with a size of a tumor diameter and is preferably increased as the tumor diameter is larger.

The step (2) may include a step of collecting the iDCs from the subject. Examples of a method for collecting the iDCs from the subject include (i) a method for collecting monocyte cells from the subject and culturing the collected monocyte cells to form iDCs, (ii) a method for obtaining iDCs from hematopoietic stem cells, and (iii) a method for obtaining iDCs by apheresis. In the first case (i), the monocyte cells can be collected and cultured by a known method. The monocyte cells can be obtained, for example, by collecting peripheral blood mononuclear cells from the subject and isolating the peripheral blood mononuclear cells. Then, the monocyte cell-depleted, T-cell enriched fraction of the peripheral blood mononuclear cells can be used to prepare activated T cells. The culture medium can vary and may be selected from those known in the art. Non-limiting examples include, but not limited to, IL-4, GM-CFS, and mixtures thereof. In the third case (iii), appropriate methods include methods that are conventionally known in the art. Further, in a case where the subject has a smaller number of tumors, it is unnecessary to prepare a large amount of CTLs. Thus, in order to easily prepare CTLs and reduce physical burden on the subject, it is preferable to collect 200 mL to 400 mL of peripheral blood from the subject and induce CTLs.

Without intending to be bound by any particular theory, it is believed that iDCs which are formed by culturing monocyte cells collected from a patient and induced comprehensive CTLs which are produced by a patient and collected from the patient, provide for an enhanced therapeutic effect when injected into the same patient as compared to iDCs and CTLs produced and obtained by other means. Induced comprehensive CTLs which have been collected, cultured and reintroduced into the same patient and iDCs which are formed from the patient's own monocyte cells which have been collected, provide improved coupling or interaction with other cells in the body of the patient.

An antibody to be administered to the subject may be at least one type of antibody that inhibits action of the inflammatory cytokine screened through the step (1). In the step (1), in a case where a plurality of inflammatory cytokines are screened, a doctor can select, as appropriate, to administer, to the subject, an antibody that inhibits action of any of the inflammatory cytokines. In a case where a plurality of inflammatory cytokines including at least one type selected from the group consisting of IL-6 and IL-5 are screened, at least one of an IL-6 blocking antibody and an anti-IL-5 blocking antibody is preferably administered as the inflammatory cytokine blocking antibody from the viewpoint of magnitude of contribution to inflammation. A description of a blocking antibody is identical to the description of the blocking antibody in [1. Composition for treating solid malignant tumor].

An amount of administration of the antibody that inhibits action of the inflammatory cytokine can be a therapeutically effective amount. The blocking antibody may be administered to the subject once or a plurality of times. In a case where the blocking antibody is administered a plurality of times, previous administration is preferably followed by subsequent administration at intervals of 30 days. An amount of the blocking antibody administered can be based on, for example, one-tenth of a determined systemic dose of each blocking antibody.

The step (2) may include a step of administering ATs to the subject after administering the iDCs and the inflammatory cytokine blocking antibody into the subject. Introducing of ATs may be immediately following or a short time after introduction of the iDCs and the inflammatory cytokine blocking antibody. Alternatively, autologous ATs may be administered from about 24 to about 72 hours following introduction of the iDCs and inflammatory cytokine blocking antibody. An amount of the ATs administered can be, for example, an intravenous drip of 1×10⁸ ATs to 2×10⁹ ATs per tumor per time. Further, the amount of the ATs administered can be adjusted as appropriate in accordance with a size of a tumor diameter and is preferably increased as the tumor diameter is larger.

The ATs can be collected from the subject, cultured, and then reintroduced to the subject. The culture medium can vary and may be selected from those known in the art. Non-limiting examples include, but are not limited to, IL-2, CD3, and mixtures thereof. Further, the ATs may be obtained from the subject by apheresis. An appropriate method of apheresis includes a method that is conventionally known in the art.

iDCs whose maturation is being induced with an adjuvant added before administration may be administered in the step (2). The adjuvant preferably includes a leukocyte cultured medium (LCM) because the leukocyte cultured medium (LCM) is suitable for a human initiated therapeutic vaccine (HITV). A description of an example of an adjuvant that can be used in an aspect of the present invention is identical to the description of the adjuvant in [1. Composition for treating solid malignant tumor].

### (Step (3))

The step (3) is a step of collecting peripheral blood mononuclear cells (PBMCs) from the subject. The PBMCs can be obtained by collecting blood from the subject and isolating the PBMCs.

In general, the PBMCs are harvested from the patient (which has been administered the autologous immature dendritic cells and an inflammatory cytokine blocking antibody) when there is a sufficient amount present in the auto-immune system of the patient, for culturing to form induced comprehensive CTLs.

In a specific embodiment in which the step (3) is carried out after the step (2), in order to provide a cytotoxic lymphocyte inducing period, it is preferable to collect the PBMCs at 2 to 6 weeks after administration of the immature dendritic cells to the subject.

Further, in a specific embodiment in which the ATs are introduced into the subject in the step (2), there is a comprehensive cytotoxic T lymphocyte (CTL) inducing period of about 2 to about 6 weeks following the introduction of the ATs. Subsequent to the comprehensive CTL inducing period, peripheral blood mononuclear cells (PBMCs) can be harvested from the patient (which has been administered the iDCs and the inflammatory cytokine blocking antibody).

### (Step (4))

The step (4) is a step of culturing the collected PBMCs and forming CTLs induced by dendritic cells. The PBMCs can be cultured by a known method. The culture medium can vary and may be selected from those known in the art. Non-limiting examples include, but are not limited to, IL-2, CD3, and mixtures thereof.

### (Step (5))

The step (5) is a step of administering, to the subject, the formed CTLs and at least one type of antibody that inhibits action of the inflammatory cytokine screened through step (1). A description of the CTLs is identical to the description of the CTLs in [1. Composition for treating solid malignant tumor].

In accordance with an aspect of the present invention, an adequate quantity and quality of induced comprehensive CTLs are administered to the patient's body, in particular, at the tumor site(s), to regress, reduce or eliminate tumor cells. It is contemplated that the quantity and quality of the induced comprehensive CTLs administered in a first introduction, e.g., intratumoral injection, may be sufficient (e.g., a therapeutically effective amount) to accomplish complete remission. However, it is also contemplated that the quantity and quality of the induced comprehensive CTLs may be insufficient and therefore, one or more additional introductions (e.g., second, third, and the like) would be needed to achieve complete remission.

An amount of the CTLs administered and an amount of the inflammatory cytokine blocking antibody administered each can be a therapeutically effective amount. In a case where the CTLs and the inflammatory cytokine blocking antibody are administered at intervals, one of the CTLs and the inflammatory cytokine blocking antibody is/are preferably administered in 90 days after administration of the other of the CTLs and the inflammatory cytokine blocking antibody. The amount of the CTLs administered can be 5×10⁸ CTLs to 5×10⁹ CTLs per tumor per time. Further, the amount of the CTLs administered can be adjusted as appropriate in accordance with a size of a tumor diameter and is preferably increased as the tumor diameter is larger.

The step (5) may be carried out only once or a plurality of times. After the step (5) is carried out once, in a case where the subject is not in complete remission due to, for example, an excessively large tumor diameter, it is preferable to carry out the step (5) a plurality of times in order to achieve complete remission. In a case where the step (5) is carried out a plurality of times, in order to induce potent comprehensive CTLs, it is preferable to repeatedly carry out the steps (3) and (4) to form the CTLs. In a case where the step (5) is carried out second and subsequent times, the CTLs and the inflammatory cytokine blocking antibody are preferably administered to partially regressed, reduced, or eliminated malignant tumors and a newly developed metastasis.

According to the therapeutic method in accordance with an aspect of the present invention, an anti-inflammatory agent other than the inflammatory cytokine inhibitory antibody may be administered to the subject in at least one of the steps (2) and (5).

Suitable anti-inflammatory agents can include those that are known in the art. The anti-inflammatory agent may be introduced simultaneously with at least one type selected from the group consisting of the iDCs, the inflammatory cytokine blocking antibody, and the CTLs, and may be introduced at predetermined time intervals from introduction of the at least one type selected from the group consisting of the iDCs, the inflammatory cytokine blocking antibody, and the CTLs. It is typical for introduction of at least one type selected from the group consisting of the iDCs, the inflammatory cytokine blocking antibody, and the CTLs and the anti-inflammatory agent to be simultaneous or substantially simultaneous, or for the elapsed time between introducing at least one type selected from the group consisting of the iDCs, the inflammatory cytokine blocking antibody, and the CTLs and the anti-inflammatory agent to be relatively short in duration.

A description of an example of the anti-inflammatory agent is identical to the description of the anti-inflammatory agent in [1. Composition for treating solid malignant tumor]. Dexamethasone is preferably administered to the subject because not only an anti-inflammatory effect but also an appetite-stimulating effect and an antiemetic effect can be expected. A description of the dexamethasone is identical to the description of the dexamethasone in [1. Composition for treating solid malignant tumor].

Further, the dexamethasone may be administered simultaneously with at least one type selected from the group consisting of the iDCs, the inflammatory cytokine blocking antibody, and the CTLs, and may be administered at predetermined time intervals before or after administration of the at least one type selected from the group consisting of the iDCs, the inflammatory cytokine blocking antibody, and the CTLs. Furthermore, the dexamethasone and at least one type selected from the group consisting of the iDCs, the inflammatory cytokine blocking antibody, and the CTLs can be combined to form a composition, and the composition can be introduced intratumorally into the patient.

An amount of the dexamethasone administered can be a therapeutically effective amount. The dexamethasone may be administered to the subject once or a plurality of times. In a case where the dexamethasone is administered a plurality of times, previous administration is preferably followed by subsequent administration at intervals of 30 days. An amount of the dexamethasone administered can be based on, for example, one-tenth to one-fourth of a determined systemic dose of dexamethasone.

Aspects of the present invention can also be expressed as follows:

A composition for treating a solid malignant tumor in accordance with a first aspect of the present invention is used so as to be administered, together with at least one type selected from the group consisting of immature dendritic cells and cytotoxic lymphocytes induced by dendritic cells, to a subject having a malignant tumor cell that produces at least one type of inflammatory cytokine selected from the group consisting of a tumor necrosis factor α, interleukin-1β, interleukin-5, interleukin-6, interleukin-8, interleukin-17, and interleukin-23, and the composition containing at least one type of antibody that inhibits action of the inflammatory cytokine.

In a second aspect of the present invention, a composition for treating a solid malignant tumor is preferably configured such that, in the first aspect, the at least one type of antibody is an antibody that inhibits action of the interleukin-6.

In a third aspect of the present invention, a composition for treating a solid malignant tumor is preferably configured such that, in the first aspect, the at least one type of antibody comprises an antibody that inhibits action of the interleukin-5 and an antibody that inhibits action of the interleukin-6.

In a fourth aspect of the present invention, a composition for treating a solid malignant tumor is preferably configured such that, in any one of the first to third aspects, the composition is intraarterially administered, intravenously administered, intramuscularly administered, intraperitoneally administered, intratumorally administered, intrapleurally administered, or subcutaneously administered.

In a fifth aspect of the present invention, a composition for treating a solid malignant tumor is preferably configured such that, in any one of the first to fourth aspects, the composition is used so as to be administered to the subject together with dexamethasone.

A kit for treating a solid malignant tumor in accordance with a sixth aspect of the present invention is used so as to be administered, together with at least one type selected from the group consisting of immature dendritic cells and cytotoxic lymphocytes induced by dendritic cells, to a subject having a malignant tumor cell that produces at least one type of inflammatory cytokine selected from the group consisting of a tumor necrosis factor α, interleukin-1β, interleukin-5, interleukin-6, interleukin-8, interleukin-17, and interleukin-23, and the kit including at least one type of antibody that inhibits action of the inflammatory cytokine.

A method of treating a solid malignant tumor in accordance with a seventh aspect of the present invention includes the following (i) step (1) and (ii) at least one selected from the group consisting of (a) step (2) and (b) steps (3) to (5) of:
(1) screening, from the group consisting of a tumor necrosis factor α, interleukin-1β, interleukin-5, interleukin-6, interleukin-8, interleukin-17, and interleukin-23, an inflammatory cytokine produced by the malignant tumor cell that a subject has;
(2) administering, to the subject, immature dendritic cells and at least one type of antibody that inhibits action of the inflammatory cytokine screened through the step (1);
(3) collecting peripheral blood mononuclear cells from the subject;
(4) culturing the collected peripheral blood mononuclear cells and forming cytotoxic lymphocytes induced by dendritic cells; and
(5) administering, to the subject, the formed cytotoxic lymphocytes and the at least one type of antibody that inhibits action of the inflammatory cytokine screened through the step (1).

In an eighth aspect of the present invention, a method is preferably configured such that, in the seventh aspect, the steps (3) to (5) are carried out after the step (2).

In a ninth aspect of the present invention, a method is preferably configured such that, in the first or second aspect, dexamethasone is further administered to the subject in at least one of the steps (2) and (5).

In a tenth aspect of the present invention, a method is preferably configured such that, in any one of the first to third aspects, the composition is administered to a malignant tumor in the subject.

A method of regressing, reducing or eliminating tumor cells in tumor tissue of a patient in accordance with an eleventh aspect of the present invention includes:
(a) introducing intratumorally a therapeutically effective amount of autologous immature dendritic cells, an anti-interleukin-6 antibody and an anti-interleukin-5 antibody, and dexamethasone into the patient; and
(b) subsequent to step (a), introducing intravenously a therapeutically effective amount of autologous activated T cells into the patient.

In a twelfth aspect of the present invention, a method is preferably configured to, in the eleventh aspect, further include:
(c) following steps (a) and (b), harvesting peripheral blood mononuclear cells from the patient;
(d) subsequent to step (c), culturing the harvested peripheral blood mononuclear cells to form induced comprehensive cytotoxic T lymphocytes; and
(e) subsequent to step (d), introducing intratumorally the induced comprehensive cytotoxic T lymphocytes, the anti-interleukin-6 antibody or the anti-interleukin-5 antibody, and dexamethasone into the patient.

In a thirteenth aspect of the present invention, a method is preferably configured to, in the twelfth aspect, further include:
(f) repeating step (e).

In a fourteenth aspect of the present invention, a method is preferably configured such that, in any one of the eleventh to thirteenth aspects, tumor cells in tumor tissue are regressed, reduced or eliminated without employing radiotherapy.

In a fifteenth aspect of the present invention, a method is preferably configured such that, in any one of the eleventh to thirteenth aspects, following the treatment steps, the patient is in remission.

In a sixteenth aspect of the present invention, a method is preferably configured such that, in the twelfth aspect, steps (c), (d) and (e) are administered when steps (a) and (b) do not result in complete remission of the patient.

In a seventeenth aspect of the present invention, a method is preferably configured such that, in the twelfth aspect, steps (c), (d) and (e) are administered to partially regressed tumor cells and/or newly developed metastasis to achieve complete remission.

In an eighteenth aspect of the present invention, a method is preferably configured such that, in the thirteenth aspect, step (f) is administered when steps (a), (b), (c), (d) and (e) do not result in complete remission of the patient.

In a nineteenth aspect of the present invention, a method is preferably configured such that, in the thirteenth aspect, step (f) is administered to partially regressed tumor cells and/or newly developed metastasis to achieve complete remission.

In a twentieth aspect of the present invention, a method is preferably configured such that, in the eleventh aspect, the introducing of the anti-interleukin-6 antibody and the interleukin-5 antibody, and dexamethasone is coincident with the introducing of the autologous immature dendritic cells.

In a twenty-first aspect of the present invention, a method is preferably configured such that, in the twentieth aspect, the autologous immature dendritic cells, the anti-interleukin-6 antibody and the interleukin-5 antibody, and dexamethasone are combined to form a composition, and the composition is introduced intratumorally into the patient.

In a twenty-second aspect of the present invention, a method is preferably configured such that, in the eleventh aspect, step (b) is administered immediately following or a short time after administering step (a).

In a twenty-third aspect of the present invention, a method is preferably configured such that, in the twenty-second aspect, step (b) is administered from about 24 to about 72 hours following step (a).

In a twenty-fourth aspect of the present invention, a method is preferably configured such that, in the twelfth aspect, steps (c), (d) and (e) are administered from about 2 to about 6 hours following steps (a) and (b).

In a twenty-fifth aspect of the present invention, a method is preferably configured such that, in the twelfth aspect, a cytotoxic T lymphocyte inducing period is administered between steps (b) and (c).

In a twenty-sixth aspect of the present invention, a method is preferably configured such that, in the twelfth aspect, a cytotoxic T lymphocyte culture period from about 2 to about 6 weeks is administered during step (d).

In a twenty-seventh aspect of the present invention, a method is preferably configured such that, in the twelfth aspect, the introducing of the anti-interleukin-6 antibody and the anti-interleukin-5 antibody, and dexamethasone is coincident with the introducing of the induced cytotoxic T cells.

In a twenty-eighth aspect of the present invention, a method is preferably configured such that, in the twelfth aspect, the induced cytotoxic T lymphocytes, the anti-interleukin-6 antibody and the anti-interleukin-5 antibody, and dexamethasone are combined to form a composition, and the composition is introduced intratumorally into the patient.

In a twenty-ninth aspect of the present invention, a method is preferably configured such that, in the twelfth aspect, culturing the CTLs is carried out in a culture medium selected from the group consisting of IL-2, CD3, and mixtures thereof.

In a thirtieth aspect of the present invention, a method is preferably configured such that, in the eleventh aspect, the tumor cells are present in metastasized tumor tissue.

In a thirty-first aspect of the present invention, a method is preferably configured such that, in the eleventh aspect, the patient is a human or a non-human mammal.

In a thirty-second aspect of the present invention, a method is preferably configured such that, in the eleventh aspect, the introducing of the autologous immature dendritic cells is in conjunction with an adjuvant.

In a thirty-third aspect of the present invention, a method is preferably configured such that, in the thirty-second aspect, the adjuvant is selected from the group consisting of lipid-based, protein-based and polysaccharides-based adjuvants, and mixtures thereof.

In a thirty-fourth aspect of the present invention, a method is preferably configured such that, in the thirty-third aspect, the adjuvant is selected from the group consisting of lymphocyte culture medium, Marignase, Agaricus, OK432, BCG, Lentinan (shiitake), Reishi, Sarunokoshikake, TNF Meshimakobu, Freund's complete or incomplete adjuvant, LPS, fatty acids, TW80, phospholipids, cytokines or a virus, and mixtures thereof.

In a thirty-fifth aspect of the present invention, a method is preferably configured such that, in the thirty-fourth aspect, the adjuvant comprises a leukocyte cultured medium (LCM).

In a thirty-sixth aspect of the present invention, a method is preferably configured such that, in the eleventh aspect, one or more of the autologous immature dendritic cells, activated T cells and cytotoxic T lymphocytes are obtained by apheresis from the patient.

A person skilled in the art would understand that the foregoing embodiment can be changed without departing from its broad inventive concept. Thus, it is understood that the present invention is not limited to a specific embodiment disclosed and that the present invention is intended to encompass a change that is within the spirit and scope of the present invention as defined by the accompanying claims.

### EXAMPLES

In Examples, a reagent the manufacturer of which is not indicated was a reagent that is commonly used in the art.

### Example 1

### (Subject)

Diagnosis: Advanced right lung cancer and multiple bone metastases
Pathology: Adenocarcinoma

### (Method for Preparing Immature Dendritic Cells)

Immature dendritic cells (iDCs) were obtained by the following method. That is, thawed monocyte nuclei (approximately 6×10⁸) were resuspended in 20 ml of an AIM-V solution and distributed in 5 mL aliquots into 44-T-75 cm² polystyrene flasks each containing 10 ral of the AIM-V solution. After culture at 37° C. for 2 hours, non-adherent cells were removed by a pipette, transferred to a conical tube, and stored to create AT cells described later. To each of flasks each containing adhesive cells, 15 mL of a DC growth solution (an AIM-V solution (CellGenix, Germany) in which 800 IU/mL of GM-CSF was added and 500 UmL of IL4 (BD Pharmingen)) was added. Culture was carried out with a flask temperature of 37° C. and 5% carbon dioxide. The growth solution was renewed on day 3, and DCs were collected on day 7 by pipetting. The collected cells were counted, resuspended in an AIM-V cryosolution containing 20% autoserum and 10% DMSO, and cryopreserved in a BICELL container (Nihon Freezer Co., Tokyo, Japan). The BICELL container is capable of stepwise freezing of cells by a programmed freezing treatment (freezing rate of 1° C./min). The cells were stored at −80° C. until being injected into a patient (for 0.5 to 3 months).

### (Method for Preparing Activated T Cells)

Activated T cells (AT) were prepared by the following method. That is, in order to generate DCs, non-adhesive T cells (approximately 6 to 9×10⁸ cells) collected after monocyte adherence were cleaned and resuspended in 20 mL of an AIM-V solution. To each of four T-225 cm² flasks each coated with an anti-CD3 antibody, 5 mL of this cell suspension and 35 mL of an AT cell solution were added (Yamazaki, T. et al, Neurol Med Chir, Tokyo, 32:255-61, 1992). Subsequently, culture was carried out with a flask temperature of 37° C. and 5% carbon dioxide. Three hours before collection, 1 μg/mL of ionomycin (Sigma, USA) was added to the solutions in order to stimulate T cells (Sato, T. et al., Cancer Immunol Immunother, 53:53-61, 2004). Since the AT cell solution is composed of an AIM-V solution in which IL-2 and autoserum area added, each flask will contain IL-2 at a final level of 1,000 IU/mL and 10% autoserum. Anti-CD antibody coating was carried out such that 10 mL of an anti-CD3 antibody (Orthoclone, OKT3 injection. Janssen Pharmaceutical, KK) at 5 μg/mL in DPBS was added to the flask and allowed to stand at room temperature for 2 hours, and then the flask was cleaned three times with 15 mL of DPBS before cells were added. The collected cells were cryopreserved and stored at −80° C. (for 0.5 to 3 months) before being injected into the patient.

### (Therapeutic Method)

First, concentrations of blood inflammatory cytokines in a subject were measured before initiation of treatment. For measurement, an ELISA assay method was used.

Next, prepared autologous immature dendritic cells were puncture-administered four times to a primary tumor in the subject in an amount of 1×10⁷ cells per tumor per time (date on which administration was carried out: Apr. 5, 2021 (first administration); Apr. 13, 2021 (second administration); May 19, 2021 (third administration); and May 20 (fourth administration)). Immediately after (24 hours to 48 hours after) the first administration, 1×10⁸ ATs per tumor were administered in drops to the subject.

Subsequently, blood collection was carried out, and concentrations of blood components were measured. The components subjected to measurement are a leukocyte (WBC), hemoglobin (Hb), a platelet (plate), total protein (TP), serum lactate dehydrogenase (LDH), and alanine transferase (ALT). Further, the concentrations of the blood inflammatory cytokines in the subject were measured again.

Next, a therapeutic composition of Example 1 was prepared by the following method on the basis of a result of measurement of the concentrations of the blood inflammatory cytokines. That is, 80 mg/4 mL of an anti-IL-6 receptor antibody Tocilizumab (available from CHUGAI PHARMACEUTICAL CO., LTD.) was used as the therapeutic composition.

Before administration of the therapeutic composition, a computed tomography (CT) scan was carried out to obtain a CT image of a lung. On Jun. 9, 2021, which was at 21 days after administration of immature dendritic cells, 80 mg (a fluid volume of 4 ml) of Tocilizumab was puncture-administered as the therapeutic composition to the primary tumor in the subject.

After administration of the therapeutic composition, a CT scan and measurement for the blood components were carried out again.

### (Result)

Table 1 shows results of measurement of the concentrations of the blood inflammatory cytokines before and after administration of the immature dendritic cells. The results shown in Table 1 have confirmed that IL-6 and IL-8 increased after administration of the immature dendritic cells. The subject in Example 1 showed a rapid tumor increase with no tumor reduction observed after administration of the autologous immature dendritic cells. Thus, it has been suggested that these inflammatory cytokines might be involved in tumor growth.

TABLE 1

| | Before administration of | | After administration of | |
|---|---|---|---|---|
| | immature dendritic cells | | immature dendritic cells | |
| Inflammatory | (measured on Mar. 19, | | (measured on May 28, | |
| cytokine | 2021) | | 2021) | |
| TNFα | 1.5 | pg/ml | 2.3 | pg/ml |
| IL-1β | 0.248 | pg/ml | 0.032 | pg/ml |
| IL-8 | 9.216 | pg/ml | 43.384 | pg/ml |
| IL-8 | 11.366 | pg/ml | 22.668 | pg/ml |

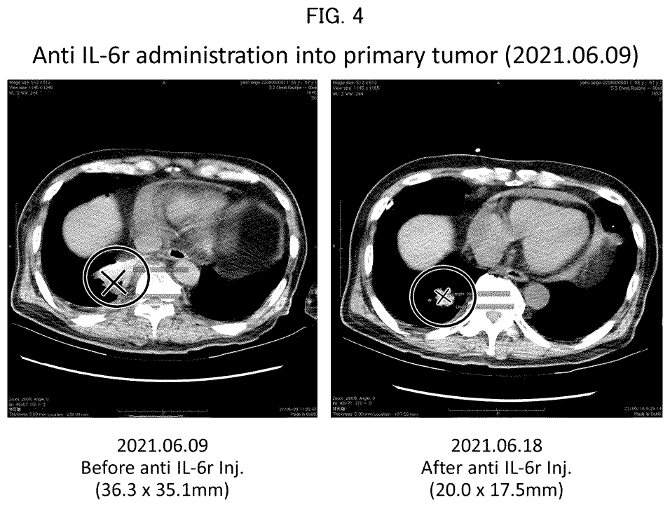

FIG. 4 shows a treatment result. In FIG. 4, the drawing on the left is a CT image obtained after administration of the iDCs and before administration of the therapeutic composition, and the drawing on the right is a CT image obtained after administration of the therapeutic composition. In FIG. 4, a treated solid malignant tumor is circled, and a cross in a circle indicates a tumor diameter. As illustrated in FIG. 4, the solid malignant tumor in the lung has been reduced by administering the anti-IL-6 receptor antibody as the therapeutic composition. This result has confirmed that the inflammatory cytokines are involved in tumor growth after administration of the autologous immature dendritic cells and that a tumor-reducing effect is obtained by inhibiting action of an inflammatory cytokine by administration of an inflammatory cytokine blocking antibody.

Table 2 shows amounts of blood components. As shown in Table 2, no change in component amount that indicates a side effect has been observed before and after administration of the therapeutic composition. Thus, no side effect that is caused by administration of the therapeutic composition has been observed.

TABLE 2

| | Before administration of | | After administration of | |
|---|---|---|---|---|
| | therapeutic composition | | therapeutic composition | |
| Blood | (measured on May 21, | | (measured on Jun. 18, | |
| component | 2021) | | 2021) | |
| WBC | 5760/μl | | 1770/μl | |
| Hb | 8.6 | g/dl | 10.5 | g/dl |
| plate | 18.3 × 10⁴/μl | | 7.9 × 10⁴/μl | |
| TP | 6.5 | g/dl | 6.0 | g/dl |
| LDH | 316 | U/L | 446 | U/L |
| ALT | 273 | U/L | 194 | U/L |

### Example 2

### (Subject)

Diagnosis: Multiple abdominal lymph node metastases caused by recurrence of rectal cancer
Pathology: Adenocarcinoma

### (Therapeutic Method)

Autologous immature dendritic cells obtained by a method similar to the method in Example 1 were puncture-administered twice to a right pelvic lymph node of a subject in an amount of 1×10⁷ cells per tumor per time (date on which administration was carried out: Dec. 20, 2021 (first administration); and Dec. 21, 2021 (second administration)).

Inflammatory cytokines in a solid malignant tumor in the subject to which immature dendritic cells had been administered were screened. Cells were collected from a rectal malignant tumor, and antibodies that specifically recognize TNFα, IL-1β, an IL-5 receptor α (IL-5ra), IL-6, IL-8, an IL-17 receptor α (IL-17ra), and an IL-23 receptor α (IL-23ra) were used to determine presence/absence of intratumoral protein expression of TNFα, IL-1β, and IL-5ra. For each of IL-5, IL-17, and IL-23, expression of the receptor but not of the cytokine itself has been confirmed. In a cell in which expression of the receptor has been successfully confirmed, it can be said that the cytokine, which is a ligand for the receptor, is also expressed. Further, a CT scan was carried out after administration of the immature dendritic cells to obtain an abdominal CT image. Furthermore, blood collection was carried out to measure concentrations of blood components. Components identical to those in Example 1 were subjected to measurement, except that aspartate aminotransferase (AST) was subjected to measurement instead of LDH.

Next, a therapeutic composition of Example 2 was prepared on the basis of results of screening. That is, a humanized anti-IL-5 receptor antibody Mepolizumab was used as the therapeutic composition.

On Apr. 26, 2022, which was at 126 days after administration of immature dendritic cells, 25 mg of the Mepolizumab was puncture-administered as the therapeutic composition to the right pelvic lymph node of the subject.

After administration of the therapeutic composition, a CT scan and measurement for the blood components were carried out again.

### (Result)

Table 3 shows the results of screening. A bar (-) in the table indicates that no protein expression has been observed. The results of screening have shown that a tumor cell produces TNFα, IL-1β, and IL-5.

TABLE 3

| TNFα | IL-1β | IL-5ra | IL-6 | IL-8 | IL-17a | IL-23a |
|---|---|---|---|---|---|---|
| 1+ | 2+ | 2+ | — | — | — | — |

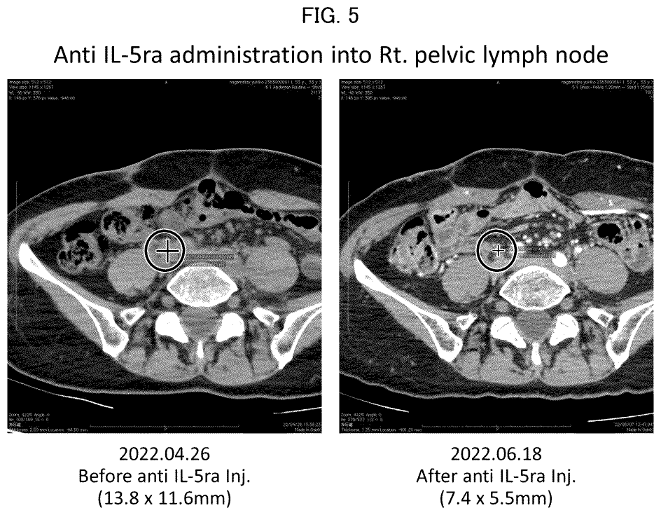

FIG. 5 shows a treatment result. In FIG. 5, the drawing on the left is a CT image obtained after administration of the iDCs and before administration of the therapeutic composition, and the drawing on the right is a CT image obtained after administration of the therapeutic composition. In FIG. 5, a treated solid malignant tumor is circled, and a cross in a circle indicates a tumor diameter. As illustrated in FIG. 5, treatment has reduced the solid malignant tumor in the right pelvic lymph node. This result has confirmed that administration of only the humanized anti-IL-5 receptor antibody as the therapeutic composition also makes it possible to obtain a sufficient tumor-reducing effect.

Table 2 shows amounts of blood components. As shown in Table 4, no change in component amount that indicates a side effect has been observed before and after administration of the therapeutic composition. Thus, no side effect that is caused by administration of the therapeutic composition has been observed.

TABLE 4

| | Before administration of | | After administration of | |
|---|---|---|---|---|
| | therapeutic composition | | therapeutic composition | |
| | (measured on Apr. 12, | | (measured on May 9, | |
| Blood component | 2022) | | 2022) | |
| WBC | 7620/μl | | 2550/μl | |
| Hb | 11.9 | g/dl | 11.6 | g/dl |
| plate | 21.6 × 10⁴/μl | | 14.6 × 10⁴/μl | |
| TP | 7.8 | g/dl | 7.4 | g/dl |
| AST | 28 | U/L | 25 | U/L |
| ALT | 18 | U/L | 15 | U/L |

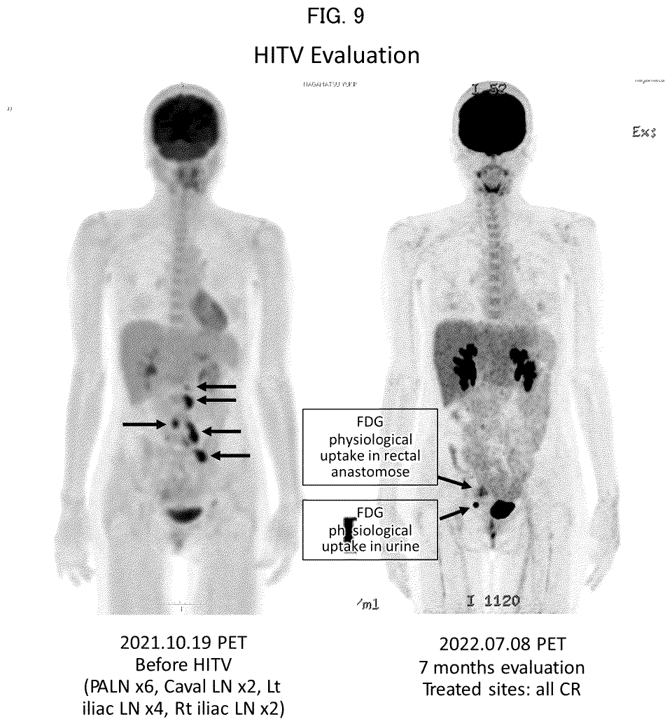

In the present test example, the treated malignant solid tumor reached complete remission (CR) at 7 months after initiation of treatment. In FIG. 9, the drawing on the left is a PET image of the subject's upper body before initiation of treatment, and the drawing on the right is a PET image of the subject's upper body at 7 months after initiation of treatment. As illustrated in FIG. 9, it has been confirmed that treated malignant solid tumors (parts indicated by arrows in the drawing on the left, i.e., malignant solid tumors at six sites in the para-aortic lymph node, at two sites in the aortic lymph node, at four sites in the left iliac lymph node, and at two sites in the right iliac lymph node) disappeared at 7 months after initiation of treatment. Parts indicated by arrows in the drawing on the right show fluorodeoxyglucose (FDG) physiological uptake in a rectal anastomosis and in urine.

### Example 3

### (Subject)

Diagnosis: Left lung cancer and multiple mediastinal lymph node metastases
Pathology: Adenocarcinoma

### (Therapeutic Method)

Inflammatory cytokines in a solid malignant tumor in a subject to which a therapeutic composition had not been administered were screened by a method similar to the method in Example 2, except that antibodies which respectively specifically recognize TNFα, IL-1β, IL-6, and IL-8 were used. Before administration of the therapeutic composition, a CT scan was carried out to obtain a CT image of a mediastinal lymph node and an aortic lymph node. Before administration of the therapeutic composition, blood collection was carried out to carry out measurement for WBC, Hb, and plate.

Next, a therapeutic composition of Example 3 was prepared on the basis of results of screening. That is, 20 mg/ml of an anti-IL-6 receptor antibody Tocilizumab (available from CHUGAI PHARMACEUTICAL CO., LTD.) was used as the therapeutic composition. Further, immature dendritic cells were prepared by a method similar to the method in Example 1.

1×10⁷ autologous immature dendritic cells per tumor and 20 mg (a fluid volume of 1 ml) of Tocilizumab were puncture-administered simultaneously as the therapeutic composition to the mediastinal lymph node of the subject (date on which administration was carried out: Mar. 8, 2022).

After administration of the therapeutic composition, a CT scan and blood collection were carried out again to measure concentrations of blood components.

### (Result)

Table 5 shows the results of screening. A bar (-) in the table indicates that no protein expression has been observed, and a blank indicates “unmeasured”. The results of screening have shown that a tumor cell produces TNFα, IL-1β, and IL-6.

TABLE 5

| | | | | | | |
|---|---|---|---|---|---|---|
| TNFα | IL-1β | IL-5ra | IL-6 | IL-8 | IL-17a | IL-23a |

| | | | |
|---|---|---|---|
| 2+ | 2+ | 1+ | — |

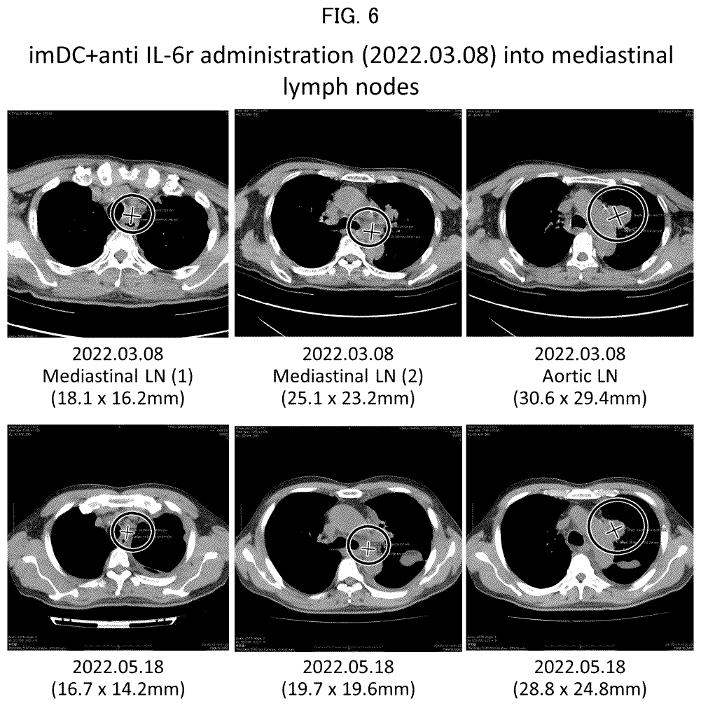

FIG. 6 shows a treatment result. In FIG. 6, the upper drawings are CT images obtained before administration of the immature dendritic cells and the therapeutic composition, and the lower drawings are CT images obtained after administration of the immature dendritic cells and the therapeutic composition. In FIG. 6, a treated solid malignant tumor is circled, and a cross in a circle indicates a tumor diameter. As illustrated in FIG. 6, treatment has reduced solid malignant tumors at two sites in the mediastinal lymph node and a solid malignant tumor in the aortic lymph node.

Table 6 shows amounts of blood components. As shown in Table 6, no change in component amount that indicates a side effect has been observed before and after administration of the immature dendritic cells and the therapeutic composition. Thus, no side effect that is caused by administration of the therapeutic composition has been observed.

TABLE 6

| | Before administration of | After administration of |
|---|---|---|
| | therapeutic composition | therapeutic composition |
| | (measured on Mar. 8, | (measured on Mar. 14, |
| Blood component | 2022) | 2022) |
| WBC | 4800/μl | 3100/μl |
| Hb | 11.1 g/dl | 11.6 g/dl |
| plate | 24.8 × 10⁴/μl | 23.1 × 10⁴/μl |

### Example 4

### (Subject)

Diagnosis: Recurrence of cervical cancer and multiple pelvic lymph node metastases
Pathology:

### (Therapeutic Method)

Autologous immature dendritic cells prepared by a method similar to the method in Example 1 were administered to five sites in a right pelvic lymph node of a subject in an amount of 1×10⁷ cells per tumor.

At 4 weeks after administration of the autologous immature dendritic cells, blood was collected from the subject, and peripheral blood mononuclear cells were isolated. Then, the peripheral blood mononuclear cells were cultured in an RPMI medium under conditions of CD3 and IL-2 through negative selection, and cytotoxic lymphocytes were obtained.

Next, inflammatory cytokines in a solid malignant tumor in a subject to which a therapeutic composition had not been administered were screened by a method similar to the method in Example 2. Before administration of the therapeutic composition, a CT scan was carried out to obtain a CT image of a pelvic lymph node. Further, blood collection was carried out to carry out measurement for WBC, Hb, plate, TP, AST, and ALT.

A therapeutic composition of Example 4 was prepared on the basis of results of screening. That is, Mepolizumab identical to that in Example 2 was used as an anti-IL-5 receptor antibody, and Tocilizumab identical to that in Example 1 was used as an anti-IL-6 receptor antibody.

2 mg of dexamethasone, the therapeutic composition (10 mg of the Mepolizumab and 5 mg of the Tocilizumab), and 5×10⁸ cytotoxic lymphocytes were puncture-administered simultaneously to five sites in the right pelvic lymph node of the subject (date on which administration was carried out: Jun. 7, 2022).

After administration of the therapeutic composition, blood collection was carried out again to measure concentrations of blood components. Further, nuclear magnetic resonance imaging (MRI) was used to obtain an MRI image of the pelvic lymph node.

### (Result)

Table 7 shows the results of screening. A bar (-) in the table indicates that no protein expression has been observed. The results of screening have shown that a tumor cell produces TNFα, IL-1β, IL-5, IL-6, IL-17, and IL-23.

TABLE 7

| TNFα | IL-1β | IL-5ra | IL-6 | IL-8 | IL-17a | IL-23a |
|---|---|---|---|---|---|---|
| 1+ | 2+ | 1+ | 1+ | — | 1+ | 1+ |

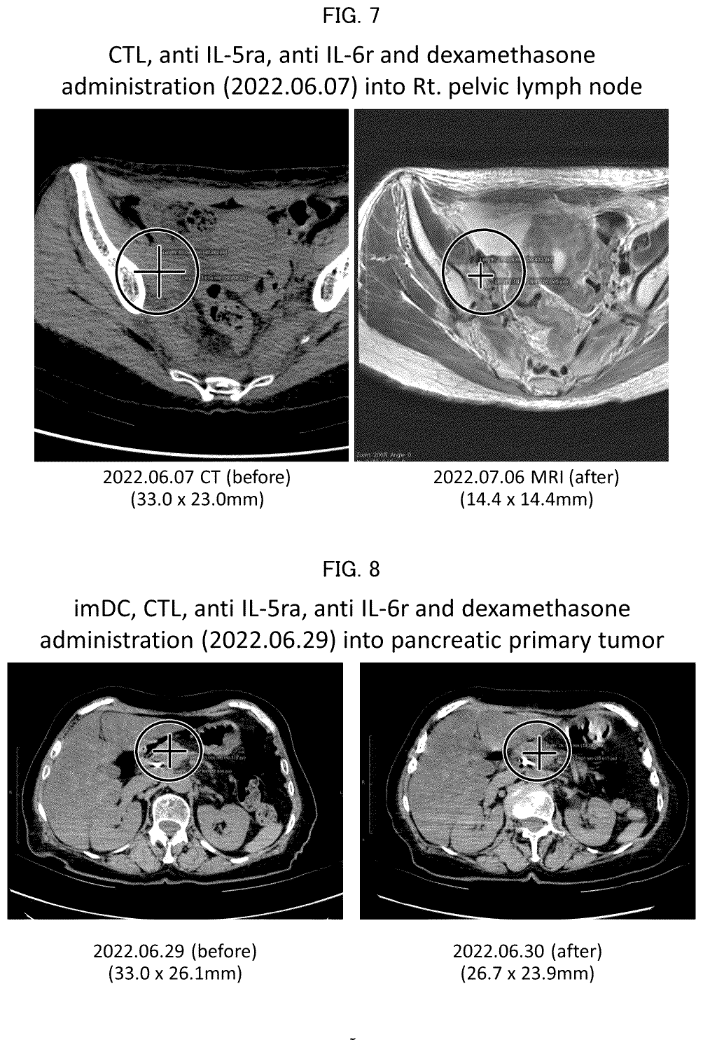

FIG. 7 shows a treatment result. In FIG. 7, the drawing on the left is a CT image obtained after administration of the iDCs and before administration of the dexamethasone, the CTLs, and the therapeutic composition, and the drawing on the right is an MRI image obtained after administration of the dexamethasone, the CTLs, and the therapeutic composition. In FIG. 7, a treated solid malignant tumor is circled, and a cross in a circle indicates a tumor diameter. As illustrated in FIG. 7, treatment has reduced the solid malignant tumor in the right pelvic lymph node.

Table 2 shows amounts of blood components. As shown in Table 8, no component amount that indicates a side effect has been observed after administration of the therapeutic composition. Thus, no side effect that is caused by administration of the therapeutic composition has been observed.

TABLE 8

| | Before administration of | | After administration of | |
|---|---|---|---|---|
| | therapeutic composition | | therapeutic composition | |
| | (measured on Jun. 7, | | (measured on Jul. 14, | |
| Blood component | 2022) | | 2022) | |
| WBC | 5920/μl | | 7980/μl | |
| Hb | 7.1 | g/dl | 7.0 | g/dl |
| plate | 23.2 × 10⁴/μl | | 29.0 × 10⁴/μl | |
| TP | 7.0 | g/dl | 6.0 | g/dl |
| AST | 21 | U/L | 15 | U/L |
| ALT | 8 | U/L | 8 | U/L |

### Example 5

### (Subject)

Diagnosis: Pancreatic cancer (inoperable)
Pathology: Adenocarcinoma

### (Therapeutic Method)

A therapeutic composition was prepared. It was impossible before initiation of treatment to establish a method for examining an inflammatory cytokine produced by a tumor in a subject in the present Example. This made it impossible to screen inflammatory cytokines produced by a tumor cell. However, since IL-5 and IL-6 were expected to be expressed, an anti-IL-5 receptor antibody Mepolizumab identical to that in Example 2 and an anti-IL-6 receptor antibody Tocilizumab identical to that in Example 1 were used as a therapeutic composition in Example 5. Further, immature dendritic cells and ATs were prepared by a method similar to the method in Example 1.

To each of three independent primary pancreatic tumors in the subject, 1×10⁷ prepared autologous immature dendritic cells per tumor and a total of 220 mg of the Tocilizumab as the therapeutic composition were puncture-administered simultaneously. Immediately thereafter, 1×10⁸ ATs per tumor were administered in drops to the subject. In Example 5, the above administration is referred to as “first administration”. At 6 months after the first administration, blood was collected from the subject, and peripheral blood mononuclear cells were isolated. Then, the peripheral blood mononuclear cells were cultured in an RPMI medium under conditions of CD3 and IL-2 through negative selection, and cytotoxic lymphocytes were obtained.

A CT scan was carried out after the first administration to obtain a CT image of the pancreas. Further, blood collection was carried out to carry out measurement for WBC, Hb, plate, TP, AST, and ALT.

On Jun. 29, 2022, which was at 7 months after the first administration, 4 mg of dexamethasone, 20 mg of the Mepolizumab and 20 mg of the Tocilizumab as the therapeutic composition, 1×10⁷ immature dendritic cells per tumor, and 5×10⁸ cytotoxic lymphocytes per tumor were puncture-administered simultaneously to each of the three tumors having been subjected to the first administration. In Example 5, the above administration is referred to as “second administration”.

After the second administration, a CT scan and blood collection were carried out again to measure concentrations of blood components. Further, it was possible at a point in time after the second administration to establish a method for examining an inflammatory cytokine produced by a tumor in the subject in the present Example. Thus, in order to identify an inflammatory cytokine produced by a tumor cell, inflammatory cytokines in a solid malignant tumor in the subject having been subjected to the second administration were screened by a method similar to that in Example 2.

### (Result)

Table 9 shows the results of screening. A bar (-) in the table indicates that no protein expression has been observed. The results of screening have shown that a tumor cell produces TNFα, IL-1β, IL-6, and IL-23.

TABLE 9

| TNFα | IL-1β | IL-5ra | IL-6 | IL-8 | IL-17a | IL-23a |
|---|---|---|---|---|---|---|
| 1+ | 2+ | — | 1+ | — | — | 1+ |

FIG. 8 shows a treatment result. In FIG. 8, the drawing on the left is a CT image obtained after the first administration, and the drawing on the right is a CT image obtained after the second administration. In FIG. 8, a treated solid malignant tumor is circled, and a cross in a circle indicates a tumor diameter. As illustrated in FIG. 8, treatment has reduced the solid malignant tumor in the pancreas.

Table 10 shows amounts of blood components. As shown in Table 10, no component amount that indicates a side effect has been observed after administration of the therapeutic composition. Thus, no side effect that is caused by administration of the therapeutic composition has been observed.

TABLE 10

| | After first | | After second | |
|---|---|---|---|---|
| | administration | | administration | |
| | (measured on Jun. 29, | | (measured on Jun. 30, | |
| Blood component | 2022) | | 2022 | |
| WBC | 4910/μl | | 6300/μl | |
| Hb | 10.7 | g/dl | 11.5 | g/dl |
| plate | 18.4 × 10⁴/μl | | 19.1 × 10⁴/μl | |
| TP | 6.5 | g/dl | 6.8 | g/dl |
| AST | 37 | U/L | 42 | U/L |
| ALT | 25 | U/L | 31 | U/L |

In view of the above, it has been made clear that the therapeutic composition in accordance with an aspect of the present invention and the therapeutic method in accordance with an aspect of the present invention make it possible to reduce a solid malignant tumor by suppressing inflammation in the solid malignant tumor.

### INDUSTRIAL APPLICABILITY

The present invention can be used to treat a solid malignant tumor.

1. (canceled)

2. (canceled)

3. (canceled)

4. (canceled)

5. (canceled)

6. (canceled)

7. A method of treating a solid malignant tumor, comprising the following (i) step (1) and (ii) at least one selected from the group consisting of (a) step (2) and (b) steps (3) to (5) of:
(1) screening, from the group consisting of a tumor necrosis factor α, interleukin-1β, interleukin-5, interleukin-6, interleukin-8, interleukin-17, and interleukin-23, an inflammatory cytokine produced by the malignant tumor cell that a subject has;
(2) administering, to the subject, immature dendritic cells and at least one type of antibody that inhibits action of the inflammatory cytokine screened through the step (1);
(3) collecting peripheral blood mononuclear cells from the subject;
(4) culturing the collected peripheral blood mononuclear cells and forming cytotoxic lymphocytes induced by dendritic cells; and
(5) administering, to the subject, the formed cytotoxic lymphocytes and the at least one type of antibody that inhibits action of the inflammatory cytokine screened through the step (1).

8. The method of claim 7, wherein the steps (3) to (5) are carried out after the step (2).

9. The method of claim 7, wherein dexamethasone is further administered to the subject in at least one of the steps (2) and (5).

10. The method of claim 7, wherein the composition is administered to a malignant tumor in the subject.

11. A method of regressing, reducing or eliminating tumor cells in tumor tissue of a patient, comprising:
(a) introducing intratumorally a therapeutically effective amount of autologous immature dendritic cells, an anti-interleukin-6 antibody and an anti-interleukin-5 antibody, and dexamethasone into the patient; and
(b) subsequent to step (a), introducing intravenously a therapeutically effective amount of autologous activated T cells into the patient.

12. The method of claim 11, further comprising:
(c) following steps (a) and (b), harvesting peripheral blood mononuclear cells from the patient;
(d) subsequent to step (c), culturing the harvested peripheral blood mononuclear cells to form induced comprehensive cytotoxic T lymphocytes; and
(e) subsequent to step (d), introducing intratumorally the induced comprehensive cytotoxic T lymphocytes, the anti-interleukin-6 antibody or the anti-interleukin-5 antibody, and dexamethasone into the patient.

13. The method of claim 12, further comprising:
(f) repeating step (e).

14. The method of claim 11, wherein tumor cells in tumor tissue are regressed, reduced or eliminated without employing radiotherapy.

15. The method of claim 11, wherein following the treatment steps, the patient is in remission.

16. The method of claim 12, wherein steps (c), (d) and (e) are administered when steps (a) and (b) do not result in complete remission of the patient.

17. The method of claim 12, wherein steps (c), (d) and (e) are administered to partially regressed tumor cells and/or newly developed metastasis to achieve complete remission.

18. The method of claim 13, wherein step (f) is administered when steps (a), (b), (c), (d) and (e) do not result in complete remission of the patient.

19. The method of claim 13, wherein step (f) is administered to partially regressed tumor cells and/or newly developed metastasis to achieve complete remission.

20. The method of claim 11, wherein the introducing of the anti-interleukin-6 antibody and the interleukin-5 antibody, and dexamethasone is coincident with the introducing of the autologous immature dendritic cells.

21. The method of claim 20, wherein the autologous immature dendritic cells, the anti-interleukin-6 antibody and the interleukin-5 antibody, and dexamethasone are combined to form a composition, and the composition is introduced intratumorally into the patient.

22. The method of claim 11, wherein step (b) is administered immediately following or a short time after administering step (a).

23. The method of claim 22, wherein step (b) is administered from about 24 to about 72 hours following step (a).

24. The method of claim 12, wherein steps (c), (d) and (e) are administered from about 2 to about 6 hours following steps (a) and (b).

25. The method of claim 12, wherein a cytotoxic T lymphocyte inducing period is administered between steps (b) and (c).

26. The method of claim 12, wherein a cytotoxic T lymphocyte culture period from about 2 to about 6 weeks is administered during step (d).

27. The method of claim 12, wherein the introducing of the anti-interleukin-6 antibody and the anti-interleukin-5 antibody, and dexamethasone is coincident with the introducing of the induced cytotoxic T cells.

28. The method of claim 12, wherein the induced cytotoxic T lymphocytes, the anti-interleukin-6 antibody and the anti-interleukin-5 antibody, and dexamethasone are combined to form a composition, and the composition is introduced intratumorally into the patient.

29. The method of claim 12, wherein culturing the CTLs is carried out in a culture medium selected from the group consisting of IL-2, CD3, and mixtures thereof.

30. The method of claim 11, wherein the tumor cells are present in metastasized tumor tissue.

31. The method of claim 11, wherein the patient is a human or a non-human mammal.

32. The method of claim 11, wherein the introducing of the autologous immature dendritic cells is in conjunction with an adjuvant.

33. The method of claim 32, wherein the adjuvant is selected from the group consisting of lipid-based, protein-based and polysaccharides-based adjuvants, and mixtures thereof.

34. The method of claim 33, wherein the adjuvant is selected from the group consisting of lymphocyte culture medium, Marignase, Agaricus, OK432, BCG, Lentinan (shiitake), Reishi, Sarunokoshikake, TNF Meshimakobu, Freund's complete or incomplete adjuvant, LPS, fatty acids, TW80, phospholipids, cytokines or a virus, and mixtures thereof.

35. The method of claim 34, wherein the adjuvant comprises a leukocyte cultured medium (LCM).

36. The method of claim 11, wherein one or more of the autologous immature dendritic cells, activated T cells and cytotoxic T lymphocytes are obtained by apheresis from the patient.
